SEQUENCE LISTING (8 sequences)

SEQ ID NO: 1 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 2 - DNA, 5765 bp, Homo sapiens
cagccgcccctcctgcggccgctgcgggggccgccgcctgacttcggacaccggccccgcacccgccaggaggggagggaaggggaggcggggagagcgacggcggggggcgggcggtggaccccgcctcccccggcacagcctgctgaggggaagagggggtctccgctcttcctcagtgcactctctgactgaagcccggcgcgtggggtgcagcgggagtgcgaggggactggacaggtgggaagatgggaatgaggaccgggcggcgggaatgttctcacttctccggattccaccgggatgcaggactctagctgcccagccgcacctgcgaagagactacacttcccgaggtgctcagcggcagcgagggcctccacgcatgcgcaccgcggcgcgctgggcggggctggatgggctgtggtgggagggttgcagcgccgcgagaaaggcgagccgggccgggggcggggaaaggggtggggcaggaacgggggcggggacggcgctggaggggcgggtcgggtaggtctcccggagctgatgtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcgggttttctttcagatattgttttgtatttacccatgaagacattgttttttggactctgcaaataggacatttcaaagatgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagttaagctggcaattaccagaacaattatgtttcttgctgtattataagaggatagctatattttatttctgaagagtaaggagtagtattttggcttaaaaatcattctaattacaaagttcactgtttattgaagaactggcatcttaaatcagccttccgcaattcatgtagtttctgggtcttctgggagcctacgtgagtacatcacctaacagaatattaaattagacttcctgtaagattgctttaagaaactgttactgtcctgttttctaatctctttattaaaacagtgtatttggaaaatgttatgtgctctgatttgatatagataacagattagtagttacatggtaattatgtgatataaaatattcatatattatcaaaattctgttttgtaaatgtaagaaagcatagttattttacaaattgtttttactgtcttttgaagaagttcttaaatacgttgttaaatggtattagttgaccagggcagtgaaaatgaaaccgcattttgggtgccattaaatagggaaaaaacatgtaaaaaatgtaaaatggagaccaattgcactaggcaagtgtatattttgtattttatatacaatttctattatttttcaagtaataaaacaatgtttttcatactgaatattaaaaaaaaaaaaaaaaaaaa

SEQ ID NO: 3 - protein, 259 aa, Homo sapiens
MARIPKTLKFVVVIVAVLLPVLAYSATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMTTSPGTPASSHYLSCTIVGIIVLIVLLIVFV

SEQ ID NO: 4 - DNA, 1512 bp, Homo sapiens
gaggccatgccccttttctgagtgcttggaagtgactgctgcaagtgacaagtgaccacgccttttcccccgcgggtataaattcagaggcgctgcgctccgattctggcagtgcagctgtgggaacctctccacgcgcacgaactcagccaacgatttctgatagatttttgggagtttgaccagagatgcaaggggtgaaggagcgcttcctaccgttagggaactctggggacagagcgccccggccgcctgatggccgaggcagggtgcgacccaggacccaggacggcgtcgggaaccataccatggcccggatccccaagaccctaaagttcgtcgtcgtcatcgtcgcggtcctgctgccagtcctagcttactctgccaccactgcccggcaggaggaagttccccagcagacagtggccccacagcaacagaggcacagcttcaagggggaggagtgtccagcaggatctcatagatcagaacatactggagcctgtaacccgtgcacagagggtgtggattacaccaacgcttccaacaatgaaccttcttgcttcccatgtacagtttgtaaatcagatcaaaaacataaaagttcctgcaccatgaccagagacacagtgtgtcagtgtaaagaaggcaccttccggaatgaaaactccccagagatgtgccggaagtgtagcaggtgccctagtggggaagtccaagtcagtaattgtacgtcctgggatgatatccagtgtgttgaagaatttggtgccaatgccactgtggaaaccccagctgctgaagagacaatgaacaccagcccggggactcctgccccagctgctgaagagacaatgaacaccagcccagggactcctgccccagctgctgaagagacaatgaccaccagcccggggactcctgccccagctgctgaagagacaatgaccaccagcccggggactcctgccccagctgctgaagagacaatgaccaccagcccggggactcctgcctcttctcattacctctcatgcaccatcgtagggatcatagttctaattgtgcttctgattgtgtttgtttgaaagacttcactgtggaagaaattccttccttacctgaaaggttcaggtaggcgctggctgagggcggggggcgctggacactctctgccctgcctccctctgctgtgttcccacagacagaaacgcctgcccctgccccaagtcctggtgtctccagcctggctctatcttcctccttgtgatcgtcccatccccacatcccgtgcaccccccaggaccctggtctcatcagtccctctcctggagctgggggtccacacatctcccagccaagtccaagagggcagggccagttcctcccatcttcaggcccagccaggcagggggcagtcggctcctcaactgggtgacaagggtgaggatgagaagtggtcacgggatttattcagccttggtcagagcagaaaaaaaaaaaaaaaaaaaa

SEQ ID NO: 5 - DNA, 23 bp, Artificial
  Oligonucleotide forward primer for Homo sapiens DCR1 gene
ttacgcgtacgaatttagttaac

SEQ ID NO: 6 - DNA, 18 bp, Artificial
  Oligonucleotide reverse primer for Homo sapiens DCR1 gene
catcaaacgaccgaaacg

SEQ ID NO: 7 - DNA, 127 bp, Artificial
  Bisulphite treated amplicon of Homo sapies DCR1 gene
ttacgcgtacgaatttagttaacgatttttgatagatttttgggagtttgattagagatgtaaggggtgaaggagcgttttttatcgttagggaattttggggatagagcgtttcggtcgtttgatg

SEQ ID NO: 8 - DNA, 127 bp, Homo sapiens
ccacgcgcacgaactcagccaacgatttctgatagatttttgggagtttgaccagagatgcaaggggtgaaggagcgcttcctaccgttagggaactctggggacagagcgccccggccgcctgatg

(19) United States
(12) Patent Application Publication
(10) Pub. No.: US 2015/0292026 A1
(43) Pub. Date: Oct. 15, 2015

(54) METHYLATION MARKERS PREDICTIVE FOR DRUG RESPONSE

(71) Applicant: MDxHEALTH SA, Sart-Tilman (Liege) (BE)

(72) Inventors: Gerrit A. Meijer, Hattem (NL); Beatriz Carvalho, Amsterdam (NL); Linda Bosch, Amstelveen (NL); Wim Van Criekinge, Waarloos (BE); Geert Trooskens, Gent (BE)

(73) Assignee: MDxHEALTH SA, Sart-Tilman (Liege) (BE)

(21) Appl. No.: 14/438,742

(22) PCT Filed: Oct. 25, 2013
(86) PCT No.: PCT/IB2013/002642

§ 371 Date: Apr. 27, 2015

Related U.S. Application Data
(60) Provisional application No. 61/718,502, filed on Oct. 25, 2012.

(51) Int. Cl. C12Q 1/68; A61K 31/4745; A61K 31/7068

(52) U.S. Cl. CPC C12Q 1/6886; A61K 31/7068; A61K 31/4745; C12Q 2600/158; C12Q 2600/154; C12Q 2600/106

(57) ABSTRACT

Disclosed are methods for detecting expression and/or aberrant methylation patterns in genes such as the gene DCR1 and their potential to diagnose or prognose a cancer or to predict drug resistance/susceptibility. More specifically, the disclosure relates to oligonucleotides, primers, probes, primer pairs and kits to detect genes such as the gene DCR1, and in particular, methylated forms of genes such as the gene DCR1. The disclosure also relates to pharmacogenetic methods to diagnose or prognose a cancer, to determine suitable treatment regimens for cancer, and to determine methods for treating cancer patients based on expression and/or aberrant methylation patterns in genes such as the gene DCR1.

### CROSS-REFERENCE TO RELATED APPLICATIONS

The present application claims the benefit of priority to U.S. Provisional Patent Application No. 61/718,502, filed on Oct. 25, 2012, the content of which is incorporated herein by reference in its entirety.

### BACKGROUND

The present disclosure relates to the detection of aberrant methylation patterns of particular genes in cancer and their potential to diagnose or prognose a cancer or to predict drug resistance/susceptibility. More specifically, the disclosure relates to oligonucleotides, primers, probes, primer pairs and kits to detect methylated forms of genes. The disclosure also relates to pharmacogenetic methods to diagnose or prognose a cancer, to determine suitable treatment regimens for cancer, and to determine methods for treating cancer patients.

### BACKGROUND

The outcome of patients with colorectal cancer (CRC) strongly depends on tumor stage at time of diagnosis. Whereas stage I CRC patients have a 5-years survival of higher then 90%, in stage IV CRC patients it just exceeds 10% (Siegel R et al., 2012. CA Cancer J Clin 2012; 62:10-29.). Chemotherapy is usually recommended for stage III and IV colorectal cancer patients. The basis of this is 5-fluorouracil-based therapy in combination with oxaliplatin or irinotecan. More recently, targeted therapies directed against vascular epithelial growth factor (VEGF) (bevacizumab) or epidermal growth factor receptor (EGFR) (cetuximab and panitumumab) have added further benefit to survival (Tol J et al., Clin Ther 2010; 32:437-53). Still, only a subset of patients benefit from these regimens, whereas patients that do not benefit still suffer from unnecessary toxicity. With the exception of KRAS mutation status that conveys resistance to epidermal growth factor receptor (EGFR)-targeted therapy (Amado R G et al. J Clin Oncol 2008; 26:1626-34; Rizzo S et al., Cancer Treat Rev 2010; 36 Suppl 3:S56-S61; Tol J et al., N Engl J Med 2009; 360:563-72), the relation between the diverse biology of CRC and treatment response is still largely unknown. Predictive biomarkers are urgently needed to identify a priori those patients that will benefit from a specific treatment versus those that will not benefit.

Several candidate predictive biomarkers have been described for colorectal cancer, of which thymidylate synthase (TS) for 5-FU, topoisomerase I (TOPI) for irinotecan and excision cross-complementing gene (ERCC1) for oxaliplatin are most promising (Jensen N F et al., Scand J Gastroenterol 2012; 47:340-55). However, these biomarkers mostly have been evaluated in single arm, non-randomized studies with limited sample sizes and results of different studies show inconsistent results, hence the predictive value of these biomarkers remain elusive (Koopman M et al., Eur J Cancer 2009; 45:1935-49).

Hypermethylated genes form a particular category of biomarkers and a number of these have been reported to have predictive value for drug response in CRC patients, such as the Werner gene (WRN) for response to Irinotecan (Agrelo R et al., Proc Natl Acad Sci USA 2006; 103:8822-7) and MGMT methylation for low risk of recurrence after treatment with capecitabine (Nagasaka T et al., Clin Cancer Res 2003; 9:5306-12.), but again inconsistent results with the same markers have been reported (Chen S P et al., Genet Test Mol Biomarkers 2009; 13:67-71; Ogino S et al., Virchows Arch 2007; 450:529-37). Hypermethylated genes are of particular interest, since DNA methylation is potentially reversible by DNA methyltransferase inhibitors, which could provide a way to restore expression of genes silenced by DNA hypermethylation and thus increase the sensitivity of tumor cells to the specific treatment modalities with which the gene is associated (Yacqub-Usman K et al., Nat Rev Endocrinol 2012; 8:486-94).

Information about how a cancer develops through molecular events could allow a clinician to get an idea of the likely course and outcome of a disease and to more accurately predict how such a cancer is likely to respond to specific therapeutic treatments. In this way, a regimen based on knowledge of the tumor sensitivity can be rationally designed and can improve management of patient care and will help identify patient populations who may particularly benefit from such approaches. It is therefore desirable to have diagnostic, prognostic, and/or predictive molecular markers that are indicative of how a tumor will respond to a therapeutic treatment such as treatment with chemotherapeutic drugs.

### SUMMARY

The present disclosure relates to methods for detecting expression or aberrant methylation patterns of particular genes in cancer and their potential use for making a diagnosis or a prognosis for a cancer patient or to be predictive for an increased, or alternatively, decreased, sensitivity of a cancer to a specific therapeutic compound or compounds. The methods further may include administering the specific therapeutic compound or compounds based on the diagnosis, prognosis, or prediction.

In particular, the disclosed methods may include: methods of predicting a clinical response to the treatment of colon cancer; methods for identifying and/or selecting a patient with colon cancer suitable for treatment; and methods of treating a cancer patient having colon cancer. The treatment may include administering to the cancer patient a topoisomerase I inhibitor, a thymidylate synthase inhibitor, and/or the combination of a topoisomerase I inhibitor and a thymidylate synthase inhibitor.

The disclosed methods may include methods of assessing, determining, and/or detecting in a sample from a patient the methylation status of a gene selected from a group consisting of DCR1, WRN, and/or regulatory regions thereof. In some embodiments of the disclosed methods, if the presence of methylation or if a higher level of methylation is detected or determined in DCR1, WRN, and/or regulatory regions thereof, the method may predict that the patient will not benefit from treatment with the topoisomerase I inhibitor or the combination of the topoisomerase I inhibitor and the thymidylate synthase inhibitor over treatment with the single agent thymidylate synthetase inhibitor or another agent. Accordingly, the methods may include administering the single agent thymidylate synthetase inhibitor to the patient and not administering the topoisomerase I inhibitor or the combination of the topoisomerase I inhibitor and the thymidylate synthase inhibitor to the patient. In further embodiments, if the presence of methylation or if a higher level of methylation is detected or determined in DCR1, WRN, and/or regulatory regions thereof, the patient will not be identified and/or selected for the treatment with the topoisomerase I inhibitor or the combination of the topoisomerase I inhibitor and the thymidylate synthase inhibitor. In even further embodiments, if the presence of methylation or if a higher level of methylation is detected or determined in DCR1. WRN, and/or regulatory regions thereof, the topoisomerase I inhibitor or the combination of the topoisomerase I inhibitor and the thymidylate synthase inhibitor will not be selected over the single agent thymidylate synthetase inhibitor treatment for administering to the patient.

In one aspect of the disclosed methods, the methods may include predicting a clinical response to treatment of colon cancer with capecitabine, irinotecan or their combination capiri in a biological sample from a patient. The methods may include: (a) assessing, determining, and/or detecting in the biological sample the methylation status of a gene selected from a group consisting of DCR1, WRN, and/or regulatory regions thereof; and (b) predicting (i) that the patient will not benefit from treatment with capiri or irinotecan over the single agent capecitabine, for example, if the presence of methylation or if a higher level of methylation is detected or determined in DCR1, WRN, and/or regulatory regions thereof; or (ii) that the patient will benefit from the treatment with capiri or irinotecan over the single agent capecitabine, for example, if the absence of methylation or if a lower level of methylation is detected or determined in DCR1, WRN, and/or regulatory regions thereof.

In another aspect of the disclosed methods, the methods may include identifying and/or selecting a patient with colon cancer suitable for treatment with capecitabine, irinotecan or their combination capiri. In this aspect, the methods may include: (a) assessing, determining, and/or detecting the methylation status of a gene selected from a group consisting of DCR1, WRN, and/or regulatory regions thereof in a biological sample obtained from the patient, and (b) identifying and/or selecting the patient for treatment with (i) capiri or irinotecan over the single agent capecitabine if the absence of methylation or if a lower level of methylation is detected or determined in DCR1, WRN, and/or regulatory regions thereof; or (ii) capecitabine rather than capiri or irinotecan if the presence of methylation or if a higher level of methylation is detected or determined in DCR1, WRN, and/or regulatory regions thereof.

In another aspect of the disclosed methods, the methods may include identifying and/or selecting a patient with colon cancer suitable for treatment with capecitabine, irinotecan or their combination capiri. In this aspect, the methods may include: (a) assessing, determining, and/or detecting expression of a gene selected from a group consisting of DCR1 and/or WRN in a biological sample obtained from the patient, and (b) identifying and/or selecting the patient for treatment with (i) capiri or irinotecan over capecitabine if the presence of expression or if a higher level of expression is detected or determined for DCR1 and/or WRN; or (ii) capecitabine over capiri or irinotecan if the absence of expression or if a lower level of expression is detected or determined for DCR1 and/or WRN.

In another aspect of the disclosed methods, the methods may include selecting a suitable treatment regimen in a patient suffering from cancer. In this aspect, the methods may include: (a) assessing, determining, and/or detecting the methylation status of the gene DCR1 and/or WRN, and/or regulatory regions thereof in a biological sample obtained from the patient; and (b) selecting (i) capiri or irinotecan over capecitabine for the treatment if the absence of methylation or if a lower level of methylation is detected or determined in DCR1 and/or WRN and/or their regulatory sequences; or (ii) capecitabine over capiri or irinotecan for the treatment if the presence of methylation or if a higher level of methylation is detected or determined in DCR1 and/or WRN and/or their regulatory regions.

In another aspect of the disclosed methods, the methods may include selecting a suitable treatment regimen in a patient suffering from cancer. In this aspect, the methods may include: (a) assessing, determining, and/or detecting expression of DCR1 and/or WRN in a biological sample obtained from the patient; and (b) selecting (i) capiri or irinotecan over capecitabine for the treatment if the presence of expression or if a higher level of expression is detected or determined for DCR1 and/or WRN; or (ii) capecitabine over capiri or irinotecan for the treatment if the absence of expression or if a lower level of expression in detected or determined for DCR1 and/or WRN.

In another aspect of the disclosed methods, the methods may include treating a cancer patient having colon cancer with capecitabine, irinotecan or their combination capiri. In this aspect, the methods may include: (a) assessing, determining, and/or detecting the methylation status of the gene DCR1 and/or WRN, and/or regulatory regions thereof in a biological sample obtained from the patient; and (b) treating the patient with (i) capiri or irinotecan rather than with single agent capecitabine if the absence of methylation or if a lower level of methylation is detected or determined in DCR1 and/or WRN and/or their regulatory regions; or (ii) capecitabine rather than capiri if the presence of methylation or if a higher level of methylation is detected or determined in DCR1 and/or WRN and/or their regulatory regions.

In another aspect of the disclosed methods, the methods may include treating a cancer patient having colon cancer with capecitabine, irinotecan or their combination capiri. In this aspect, the methods may include: (a) assessing, determining, and/or detecting expression of DCR1 and/or WRN in a biological sample obtained from the patient; and (b) treating with (i) capiri or irinotecan rather than capecitabine if the presence of expression or if a higher level of expression is detected or determined for DCR1 and/or WRN; or (ii) capecitabine rather than capiri if the absence of expression or if a lower level of expression is detected or determined for DCR1 and/or WRN.

In another aspect of the disclosed methods, the methods may include: (a) requesting a test providing results of an analysis to determine the methylation status of a gene selected from a group consisting of DCR1. WRN, and/or their regulatory regions in a biological sample obtained from a patient; and (b) administering capecitabine, irinotecan, and/or capiri based on the results of the test. For example, the methods may include: (a) requesting a test providing results of an analysis to determine whether a gene selected from a group consisting of DCR1, WRN, and/or their regulatory regions are nonmethylated, methylated, or hypermethylated in a biological sample obtained from a patient and/or whether a gene selected from a group consisting of DCR1, WRN, and/or their regulatory regions are exhibiting a lower level of methylation or a higher level of methylation in a biological sample from a patient (for example, relative to a control); and (b) treating the patient with (i) capiri or irinotecan rather than capecitabine if the gene is nonmethylated in the biological sample obtained from the patient and/or if the gene is exhibiting a lower level of methylation (or hypermethylation) in the biological sample from the patient (for example, relative to a control); or (ii) capecitabine rather than capiri if the gene is methylated (or hypermethylation) in the biological sample obtained from the patient and/or if the gene is exhibiting a higher level of methylation (or hypermethylation) in the biological sample from the patient (for example, relative to a control). In this later instance capecitabine may be administered alone or may be administered as a combination drug that does not include irinotecan, such as capox or capox-B.

In another aspect of the disclosed methods, the methods may include: (a) requesting a test providing results of an analysis to determine expression status of a gene selected from a group consisting of DCR1 and/or WRN in a biological sample obtained from a patient; and (b) administering capecitabine, irinotecan, and/or capiri based on the results of the test. For example, the methods may include: (a) requesting a test providing results of an analysis to determine whether a gene selected from a group consisting of DCR1 and/or WRN is expressed or is not expressed in a biological sample obtained from a patient and % or whether a gene selected from a group consisting of DCR1 and/or WRN is expressed at a lower level or is expressed at a higher level in a biological sample from a patient (for example, relative to a control); and (b) treating the patient with (i) capiri or irinotecan if the gene is expressed in the biological sample obtained from the patient and/or if the gene is expressed at a higher level in the biological sample from the patient (for example, relative to a control); or (ii) capecitabine if the gene is not expressed in the biological sample obtained from the patient and/or if the gene is expressed at a lower level in the biological sample from the patient (for example, relative to a control). In this later instance capecitabine may be administered alone or may be administered as a combination drug that does not include irinotecan, such as capox or capox-B.

Also disclosed herein are uses of capecitabine, irinotecan or their combination capiri in treating cancer in a patient, wherein the patient has been selected for treatment on the basis of the methods disclosed herein for detecting or determining the methylation status of a gene selected from a group consisting of DCR1, WRN, and/or their regulatory regions. For example, disclosed herein is the use of capecitabine to treat cancer in a patient where a gene selected from a group consisting of DCR1, WRN, and/or their regulatory regions is methylated in a biological sample obtained from the patient and/or where a gene selected from a group consisting of DCR1, WRN, and/or their regulatory regions is exhibiting a higher level of methylation in a biological sample from the patient (for example, relative to a control). In another example, disclosed herein is the use of capiri or irinotecan to treat cancer in a patient where a gene selected from a group consisting of DCR1 WRN, and/or their regulatory regions is nonmethylated in a biological sample obtained from the patient and/or where a gene selected from a group consisting of DCR1, WRN, and/or their regulatory regions is exhibiting a lower level of methylation in a biological sample from the patient (for example, relative to a control).

Also disclosed herein are uses of capecitabine, irinotecan or their combination capiri in treating cancer in a patient, wherein the patient has been selected for treatment on the basis of the methods disclosed herein for detecting or determining the expression status of a gene selected from a group consisting of DCR1 and/or WRN. For example, disclosed herein is the use of capecitabine to treat cancer in a patient where a gene selected from a group consisting of DCR1 and/or WRN is not expressed in a biological sample obtained from the patient and/or where a gene selected from a group consisting of DCR1 and/or WRN is exhibiting a lower level of expression in a biological sample from the patient (for example, relative to a control). In another example, disclosed herein is the use of capiri or irinotecan to treat cancer in a patient where a gene selected from a group consisting of DCR1 and/or WRN is expressed in a biological sample obtained from the patient and/or where a gene selected from a group consisting of DCR1 and/or WRN is exhibiting a higher level of expression in a biological sample from the patient (for example, relative to a control).

Also disclosed herein are kits for assessing methylation in a test sample. The kit optionally may include a reagent that (a) modifies methylated cytosine residues but not non-methylated cytosine residues, or that (b) modifies non-methylated cytosine residues but not methylated cytosine residues. The kit also may include a pair of oligonucleotide primers that specifically hybridizes under amplification conditions to the methylated gene or regulatory regions thereof following treatment with a reagent, which gene is selected from a group consisting of DCR1 and/or WRN.

Also provided are methods of detecting cancer comprising determining the methylation status or expression of a gene of interest (e.g., DCR1 and/or WRN) in a sample obtained from a patient (e.g., a biological sample obtained from a patient suspected of having colon cancer), wherein the methylation status or expression is assessed using methods disclosed herein.

These and other embodiments which will be apparent to those of skill in the art upon reading the specification.

### BRIEF DESCRIPTION OF THE FIGURES

FIG. 1: Study Design. Patients were selected based on similar clinical characteristics compared to all patients in the Dutch pectiabine, rinotecan, and Qxaliplatin “CAIRO” in Advanced Colorectal Cancer study. For PFS analysis, only patients that received ≧3 cycli of a certain treatment-line or ≧2 cycli when cause of death was progressive disease were included. For OS analysis, all patients were included.

FIG. 2: Progression-free survival for patients with methylated (dashed line) and unmethylated DCR1 (solid line) after treatment with first line capecitabine (A) and after treatment with first line capiri (B)

FIG. 3: Progression-free survival after first line capecitabine (solid line) and first line capiri (dashed line) treatment in patients of the discovery set with unmethylated DCR1(A) and methylated DCR1 (B).

FIG. 4: Progression-free survival after first line capecitabine (solid line) and first line capiri (dashed line) treatment in patients of the validation set with unmethylated DCR1(A) and methylated DCR1 (B).

FIG. 5: Relative DCR1 mRNA expression: measured in 13 CRC cell lines (A); in HCT116 following treatment with 5-aza-2′-deoxycytidine (B); correlation between DCR1 methylation and mRNA expression in 78 CRC tumors (C).

FIG. 6: Study design of the screen to identify genes whose methylation status correlates to drug response (GI50) in the cells selected from the NCI database.

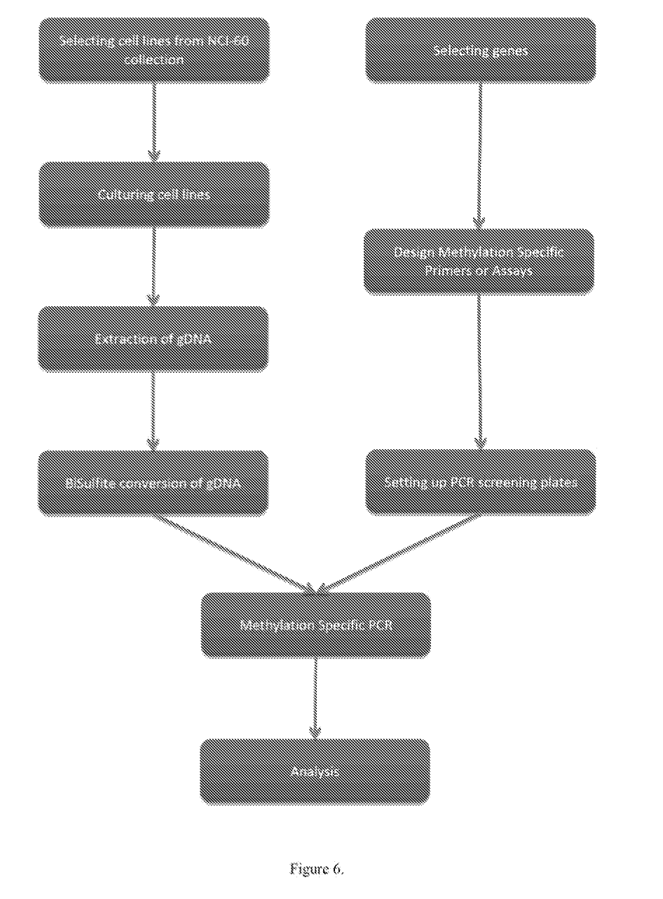

FIG. 7: Plot of progression free survival (PFS) versus time for patients treated with capecitabine. (Hazard Rations (HR)=1.4 (95% CI 0.9-2.0), p=0.1).

### DETAILED DESCRIPTION

Using a systematic approach to identify methylation regulated marker genes in cell conversion, the inventors have identified genes whose methylation status and/or expression levels may be utilized to make a diagnosis and/or prognosis of a cancer patient or to be predictive for an increased, or alternatively, decreased, sensitivity to a specific therapeutic compound or a combination of compounds. Assays assessing the methylation status or expression of the identified genes find their application in the diagnosis and/or prognosis of cancer and the treatment of patients with pharmaceutical compounds.

The present study aimed to identify DNA methylation markers with predictive or prognostic value for response to chemotherapy. For this purpose, a candidate gene approach was used and DNA methylation was analyzed on primary CRC tissues of a sub-group of patients from the Dutch Capecitabine, Irinotecan, and Oxaliplatin “CAIRO” in Advanced Colorectal Cancer study, a randomized phase III study to assess the sequential or combination treatment of advanced colorectal cancer patients with capecitabine, irinotecan, and oxaliplatin. In total 2 genes with strong predictive and/or prognostic value were identified: DCR1 and WRN.

The methods disclosed herein may be performed: for predicting a clinical response to the treatment of colon cancer; for identifying and/or selecting a patient with colon cancer suitable for treatment; and/or for treating a cancer patient having colon cancer with a topoisomerase I inhibitor, a thymidylate synthase inhibitor, and/or the combination of a topoisomerase I inhibitor and a thymidylate synthase inhibitor. The disclosed methods may include assessing, determining, and/or detecting in a sample from a patient the methylation status of a gene selected from a group consisting of DCR1, WRN, and/or regulatory regions thereof. Based upon the detection or determination of the presence or absence of methylation and/or a higher or lower level of methylation of DCR1, WRN, the method may predict: whether the patient will benefit from treatment with the topoisomerase I inhibitor (e.g., administered as a combination of the topoisomerase I inhibitor and the thymidylate synthase inhibitor) versus treatment with the single agent thymidylate synthetase inhibitor; or whether the patient will benefit from the treatment with the single agent thymidylate synthetase inhibitor over treatment with the topoisomerase I inhibitor (e.g., administered as a combination of the topoisomerase I inhibitor and the thymidylate synthase inhibitor).

As shown herein, methylation (or hypermethylation) of a gene can predict the response to combined topoisomerase I inhibitor and thymidylate synthase inhibitor treatment in patients with metastatic colorectal cancer. For instance, patients with DCR1 methylated in their tumor do not benefit from the addition of the topoisomerase I inhibitor to the thymidylate synthase inhibitor, in strong contrast to patients with unmethylated DCR1 in their tumor. Accordingly, the presently disclosed methods may include assessing, determining, and/or detecting the methylation status or expression of a gene in a biological sample obtained from the patient or patient with cancer. The gene under investigation is chosen from the group consisting of DCR1, WRN, and/or their regulatory regions. The presence of methylation (or hypermethylation) or a higher level of methylation of DCR1, WRN, and/or their regulatory regions is indicative that the patient will not benefit from treatment with the combination of the topoisomerase I inhibitor and the thymidylate synthase inhibitor over treatment with the single agent thymidylate synthetase inhibitor alone. Conversely, the absence of methylation (or hypermethylation) or a lower level of methylation (or hypermethylation) of DCR1, WRN, and/or their regulatory regions is indicative that the patient will benefit from treatment with the combination of the topoisomerase I inhibitor and the thymidylate synthase inhibitor over treatment with the single agent thymidylate synthetase inhibitor alone.

The likelihood that a patient will not benefit from treatment with the combined topoisomerase I inhibitor and the thymidylate synthase inhibitor over the single agent thymidylate synthase inhibitor alone is high in a situation where the presence of methylation (or hypermethylation) or a higher level of methylation (or hypermethylation) of DCR1, WRN, and/or their regulatory regions is detected or determined. In that case, the patient is not selected for treatment with the topoisomerase I inhibitor and the thymidylate synthase inhibitor combination and one or more alternative drugs may be more beneficial for the treatment of the cancer patient. The likelihood that a patient will benefit from the treatment with the topoisomerase I inhibitor and the thymidylate synthase inhibitor combination over the single agent thymidylate synthase inhibitor is high in a situation where the absence of methylation (or hypermethylation) or a lower level of methylation (or hypermethylation) lack of DCR1, WRN, and/or their regulatory regions is detected or determined. In that case, patients will benefit from addition of the topoisomerase I inhibitor to the single agent thymidylate synthase inhibitor. Because hypermethylation is inversely correlated with expression of the gene concerned, in particular DCR1, patients will benefit from treatment with the topoisomerase I inhibitor and the thymidylate synthase inhibitor combination over the single agent thymidylate synthase inhibitor in a situation where expression (or a higher level of expression relative to a control) of DCR1 is detected or determined.

As contemplated herein, the thymidylate synthase inhibitor preferably is a thymidylate synthase inhibitor prodrug. Suitable thymidylate synthase inhibitor prodrugs may include, but are not limited to capecitabine. As contemplated herein, suitable topoisomerase I inhibitors may include, but are not limited to irinotecan. As contemplated herein, the combination drug including the topoisomerase I inhibitor and the thymidylate synthase may include, but is not limited to a combination of capecitabine and irinotecan, also called capiri. Combination drugs comprising capecitabine, but not comprising irinotecan, may include, but are not limited to capox and capox-B.

The disclosed methods may include methods of predicting a clinical response to treatment of colon cancer with capecitabine, irinotecan or their combination, capiri, the methods comprising: (a) obtaining a biological sample from a patient; (b) assessing, determining, and/or detecting in the sample the methylation status of a gene selected from a group consisting of DCR1, WRN, and/or regulatory regions thereof, and (c) determining that the patient will not benefit from the treatment with capiri or irinotecan over the single agent capecitabine if the presence of methylation or a higher level of methylation is detected or determined in DCR1. WRN, and/or regulatory regions thereof. In that case, other therapies, such as capecitabine alone or capox-based therapies, may provide an alternative for patients with DCR1 methylated CRC. The methods therefore may include administering a capox-based therapy to a CRC patient exhibiting methylation in DCR1 or the regulatory regions of DCR1 in a biological sample from the CRC patient. For example, the methods may include administering capox or capox-B to a CRC patient exhibiting methylation in DCR1 or the regulatory regions of DCR1 in a biological sample from the CRC patient.

The disclosed methods may include methods of predicting a clinical response to treatment of colon cancer with capecitabine, irinotecan or their combination, capiri, the methods comprising: (a) obtaining a biological sample from a patient; (b) assessing, determining, and/or detecting in the sample the methylation status of a gene selected from a group consisting of DCR1, WRN, and/or regulatory regions thereof, and (c) determining that the patient will benefit from the treatment with capiri or irinotecan over the single agent capecitabine if the absence of methylation or a lower level of methylation is detected or determined in DCR1, WRN, and/or regulatory regions thereof.

As shown in the example section, hypermethylation is associated with decreased gene expression. Treatment of cell lines showing gene methylation with the demethylating agent 5-aza-2′-deoxycytidine (DAC) resulted in significantly increased gene expression. Accordingly, the disclosed methods may include predicting a clinical response to treatment of colon cancer with capecitabine, irinotecan or their combination, capiri comprising: (a) obtaining a biological sample from a patient; (b) assessing, determining, and/or detecting in the sample expression of the gene DCR1 and/or WRN; and (c) determining that the patient will not benefit from the treatment with capiri or irinotecan over the single agent capecitabine if the absence of expression or if a lower level of expression of DCR1 and/or WRN is determined or detected. In that case, other therapies, such as capecitabine alone or capox-based therapies, may provide an alternative for CRC patients not expressing DCR1 or expressing a low level of DCR1. The methods therefore may include administering a capox-based therapy to a CRC patient not expressing DCR1 or exhibiting a low level of expression of DCR1 in a biological sample from the CRC patient. For example, the methods may include administering capox or capox-B to a CRC patient not expressing DCR1 or exhibiting a low level of expression of DCR1 in a biological sample from the CRC patient.

Conversely, the disclosed methods may include predicting a clinical response to treatment of colon cancer with capecitabine, irinotecan or their combination, capiri, the methods comprising: (a) obtaining a biological sample from a patient; (b) assessing, determining, and/or detecting in the sample expression of the gene DCR1 and/or WRN; and (c) determining that the patient will benefit from the treatment with capiri or irinotecan over the single agent capecitabine if the presence of expression or if a higher level of expression of DCR1 and/or WRN is determined or detected.

In another aspect, the methods may include predicting the likelihood of successful treatment with capiri or irinotecan in a cancer patient, the methods comprising: (a) assessing, determining, and/or detecting in a biological sample from the patient: (i) the methylation status of a gene chosen from the group consisting of DCR1, WRN, and/or regulatory regions thereof, or (ii) the expression of a gene selected from a group consisting of DCR1 and/or WRN: and (b) predicting a successful treatment with capiri or irinotecan: (i) where DCR1, WRN and/or regulatory regions thereof are nonmethylated or are methylated at a lower level; or (ii) where DCR1 and/or WRN are expressed or are expressed at a higher level.

“Cancer” refers to the presence of cells possessing characteristics typical of cancer-causing cells, such as uncontrolled proliferation, immortality, metastatic potential, rapid growth and proliferation rate, and certain characteristic morphological features. Particular cancer types include those selected from breast, colon, leukemia, lung, melanoma, ovarian, prostate and renal cancer. Most preferably, the cancer involved is a colon or colorectal cancer. “Colon cancer,” also called colorectal cancer or bowel cancer, is defined to include cancerous growths in the colon, rectum and appendix.

“Patient” may be utilized interchangeably with “subject” or “individual” and is intended to include humans and non-humans. A “patient” may include a human having or suspected of having a cancer, such as colorectal cancer (CRC), i.e., a “CRC patient.”

By “methylation status” is meant the level of methylation of cytosine residues (found in CpG pairs) in the gene of interest which are relevant to the regulation of gene expression. Methylation of a CpG island at a promoter usually prevents expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns. All of these regions can be assessed to determine their methylation status, as appropriate. The levels of methylation of the gene of interest are determined by any suitable means in order to reflect whether the gene is likely to be downregulated or not. Levels of methylation or hypermethylation may be determined relative to a control and may reflect “lower” levels relative to the control or may reflect “higher” levels relative to the control.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. A methylation status can thus be expressed in terms of a higher or a lower level of methylation at one or a plurality of CpG dinucleotides within a DNA sequence.

By “expression status” is meant the level of mRNA and/or translated protein associated with a gene in a biological sample. “Expression status” may be assessed qualitatively where mRNA and/or translated protein are detected above background level. “Expression status” may be assessed relative to a control (e.g., a negative control, a positive control, or relative to expression of a so-called “housekeeping genes”).

“Diagnosis” is defined to me determination or identification of a disease or disorder in a patient, or the lack thereof. “Diagnosis” may include determining or identifying a stage of a disease or disorder in a patient. “Prognosis” is defined to include an assessment or prediction of the probable course, outcome, recovery or survival from a disease. Most physicians give a prognosis based on statistics of how a disease acts in studies on the general population. Prognosis can vary with cancer depending on several factors, such as the stage of disease at diagnosis, type of cancer, and even gender.

“Overall survival” is a term that denotes the chances of staying alive for a group of individuals suffering from a cancer. It denotes the percentage of individuals in the group who are likely to be alive after a particular duration of time. At a basic level, the overall survival is representative of cure rates. A Kaplan-Meier analysis allows estimation of survival over time, even when patients drop out or are studied for different lengths of time.

“Test samples” for diagnostic, prognostic, or personalized medicinal uses may be obtained from surgical samples, such as biopsies or fine needle aspirates, from paraffin embedded tissues, from frozen tumor tissue samples, from fresh tumor tissue samples, from a fresh or frozen body fluid, for example. Preferably, the test sample is obtained from a human patient. Most preferably the sample is taken from a patient suspected of being tumorigenic and contains cells derived from colon or colorectal tissue or nucleic acids from such cells. However, any other suitable test samples (e.g. bodily fluids such as blood, stool, and the like) in which the methylation status of a gene of interest can be determined to indicate the presence of cancer are contemplated herein.

A treatment treats a problem, and may lead to complete recovery, but treatments more often ameliorate a problem only for as long as the treatment is continued. “Successful treatment” is defined to include complete recovery, significant tumor regression, prevention of metastasis and an increase in survival. Increase in survival includes increased survival time and/or improved survival rates. Therefore, use of combinations of any one or more of the listed therapeutic agents may be required to obtain longer survival. Improved alleviation of symptoms may also be considered as “successful treatment.” “Likelihood of successful treatment” means the probability that treatment of cancer using any one or more of the listed therapeutic agents will be successful.

“Resistance” is defined as a reduced probability that treatment of cancer will be successful using any one or more of the listed therapeutic agents and/or that higher dose or other therapeutic agents will be required to achieve a therapeutic effect. The presently disclosed methods may be utilized to identify cancer (e.g. colorectal cancer) that is resistant to treatment with irinotecan. For example, in the disclosed methods, irinotecan-resistant colorectal cancer may be identified in a patient where DCR1, WRN, and/or their regulatory regions are methylated or hypermethylated in a patient sample.

The disclosed methods may include detecting methylation or hypermethylation of a nucleic acid of a gene. Preferably, the nucleic acid is DNA and is obtained from a test sample isolated from a patient suspected of being tumorigenic. The nucleic acid may be obtained from the gene DCR1, WRN, and/or their regulatory regions. “WRN” and “DCR1” are the standard nomenclature as approved by the Human Genome Organization, although DCR1 may alternatively be referred to as “TNFRSF10C.” At least one of the genes WRN or DCR1 is a gene of interest for use in the methods and assays as disclosed herein.

“WRN” Werner protein (Accession number: NM_{—}000553.4) is a member of the RecQL DNA helicase family. It also functions as a 3′ to 5′ exonuclease, and is involved in telomere maintenance. Mutations in WRN lead to a genetic instability syndrome, Werner syndrome, which is manifested by premature aging and tumor predisposition. Werner syndrome cells exhibit early replicative senescence and cell proliferation defects, increased sensitivity to DNA damaging agents, and genetic instability [Ozgenc et al, GenomeDis, 2006]. In sporadic neoplasia, WRN often shows loss of heterogeneity, but mutations have not been found. Instead, epigenetic inactivation by DNA hypermethylation is found in several tumor types, including CRCs [Nosho 2009; Kawasaki 2008; Ogino 2007; Agrelo et al, PNAS, 2006]. The amino acid sequence of the WRN protein is provided herein as SEQ ID:1 and the nucleic acid sequence of the WRN gene is provided as SEQ ID NO:2, based on the information deposited at Accession number: NM_{—}000553.4.

“DCR1” Decoy receptor I (Accession number: NM_{—}003841), is a decoy receptor for tumor necrosis factor (TNF) related apoptosis inducing ligand (TRAIL). It is able to bind TRAIL, but fails to induce apoptosis since it lacks an intracellular death domain. It thereby functions as an anti-apoptosic factor of the extrinsic apoptosis pathway [ref]. DCR1 is frequently downregulated in several cancer types for which DNA hypermethylation has been associated [Shivapurkar N, 2004, ref]. DNA methylation in CRC has not been reported so far. The amino acid sequence of the DCR1 protein is provided herein as SEQ ID:3 and the nucleic acid sequence of the WRN gene is provided as SEQ ID NO:4, based on the information deposited at Accession number: NM_{—}003841.

The genes encompass not only the particular sequences found in the publicly available database entries, but also encompass variants of these sequences. Variant sequences may have at least 90%, at least 91%, at least 92%, at least 93%, at least 94%, at least 95%, at least 96%, at least 97%, at least 98%, or at least 99% identity to sequences in the database entries or sequence listing. Computer programs for determining percent identity are available in the art, including Basic Local Alignment Search Tool (BLAST5) available from the National Center for Biotechnology Information. The genes are available as indicated hereafter. Variant sequences may encode variant proteins and may include truncated forms of the proteins (i.e., truncated forms having N-truncations, C-truncations, or both). Variant proteins may result from translation of alternatively spliced mRNAs. Variant proteins also may comprise post-translational modifications. Preferably, the variant proteins have one or more biological activities of the wild-type proteins. For example, a variant WRN protein may have helicase activity or 3′→5′ exonuclease activity, and a variant DCR1 protein may have TNF binding activity.

As discussed, the absence of methylation (or hypermethylation) or a lower level of methylation (or hypermethylation) of DCR1, WRN, and/or their regulatory regions indicates a favorable response to treatment with capiri or irinotecan. In that case, the patient is identified or selected for treatment with capiri or irinotecan over capecitabine. Accordingly, the disclosed methods include identifying and/or selecting a patient with cancer suitable for treatment with capecitabine, irinotecan or their combination comprising assessing, determining, and/or detecting in a test sample of the patient the methylation status of the gene DCR1, WRN, and/or their regulatory regions, and/or regulatory regions thereof. The cancer patient is selected for treatment with capiri or irinotecan over capecitabine in a situation where absence of methylation (or hypermethylation) of DCR1, WRN and/or their regulatory sequences is observed or where a lower level of methylation (or hypermethylation) of DCR1, WRN and/or their regulatory sequences is observed. CRC patients that do not benefit from adding irinotecan to capecitabine therapy should not suffer from unnecessary toxicity. Therefore, the opposite scenario also applies and the cancer patient will not be selected for treatment with capiri or irinotecan over the single agent capecitabine in a situation where the presence of methylation (or hypermethylation) of DCR1, WRN and/or their regulatory sequences is observed or where a higher level of methylation (or hypermethylation) of DCR1, WRN and/or their regulatory sequences is observed. In that case, other therapies, such as capecitabine alone or combination drugs such as capox-based may provide an alternative for patients with DRC1 methylated CRC. These may include treatment with capox and/or capox-B.

In another aspect, the disclosed methods may include identifying and/or selecting a patient with colon cancer suitable for treatment with capecitabine, irinotecan or their combination capiri. The methods may include: (a) obtaining a biological sample from the patient; (b) assessing, determining, and/or detecting in the sample the methylation status of a gene selected from a group consisting of DCR1, WRN, and/or regulatory regions thereof; and (c) identifying and/or selecting the patient for treatment with capiri or irinotecan over the single agent capecitabine if the absence of methylation (or hypermethylation) of the gene and/or their regulatory sequences is determined or detected, or if a lower level of methylation (or hypermethylation) of the gene and/or their regulatory sequences is determined or detected. Preferably, the patient is selected for first-line capiri treatment. The methods further may include administering capiri or irinotecan treatment to the patient thus identified and/or selected. Other methods may include: (a) obtaining a biological sample from the patient; (b) assessing, determining, and/or detecting in the sample the methylation status of a gene selected from a group consisting of DCR1, WRN, and/or regulatory regions thereof: and (C) identifying and/or selecting the patient for treatment with capecitabine or another agent over capiri or irinotecan if the presence of methylation (or hypermethylation) of the gene and/or their regulatory sequences is determined or detected, or if a higher level of methylation (or hypermethylation) of the gene and/or their regulatory sequences is determined or detected. The methods further may include administering capecitabine treatment or another agent to the patient thus identified and/or selected. Other agents may include, but are not limited to, capox treatment and/or capox-B treatment.

“Capecitabine” is an orally-administered chemotherapeutic agent used in the treatment of metastatic breast and colorectal cancers. Capecitabine is a prodrug, that is enzymatically converted to 5-fluoroucil in the tumor, where it inhibits DNA synthesis and slows growth of tumor tissue. The activation of capecitabine follows a pathway with three enzymatic steps and two intermediary metabolites, 5′-deoxy-5-fluorocytidine (5′-DFCR) and 5′-deoxy-5-fluorouridine (5′-DFUR), to form 5-fluorouracil.

“Irinotecan” is a drug used for the treatment of cancer such as colon cancer, in particular in combination with other chemotherapy agents. Irinotecan is a topoisomerase I inhibitor, which prevents DNA from unwinding. In chemical terms, it is a semisynthetic analogue of the natural alkaloid camptothecin.

“Capiri” is a combination drug comprising Irinotecan and Capecitabine and is used for the treatment of colon cancer.

In the methods disclosed herein where capecitabine is administered rather than capiri or irinotecan, capecitabine may be administered as a combination drug other than capiri. Suitable combination drugs other than capiri may include capox and capox-B. “Capox” is a combination drug comprising Capecitabine and oxaliplatin. “Capox-B” is a combination drug comprising Capecitabine, oxaliplatin and bevacizumab.

The methods disclosed herein may be utilized to select a suitable course of treatment for a patient. In the methods, the absence of methylation (or the absence of hypermethylation) or a lower level of methylation (or a lower level of hypermethylation) of DCR1, WRN, and/or their regulatory regions indicates that a combination of irinotecan and capecitabine may be beneficially administered over the single agent capecitabine. Thus, the methods may include selecting a suitable treatment regimen, or a combination treatment regimen, in a patient suffering from cancer, the method including: (a) obtaining a biological sample from the patient; (b) assessing, determining and/or detecting the methylation status of the gene DCR1, WRN, and/or their regulatory regions, and/or regulatory regions thereof in the biological sample; and, (c) selecting capiri or irinotecan over the single agent capecitabine for the treatment if the absence of methylation (or hypermethylation) of the gene and/or their regulatory sequences is determined or detected, or if a lower level of methylation (or hypermethylation) of the gene and/or their regulatory sequences is determined or detected. Preferably, the patient is selected for first-line capiri treatment. The methods further may include administering the selected capiri or irinotecan treatment to the patient. Other methods for selecting a suitable treatment regimen, or a combination treatment regimen, in a patient suffering from cancer may include: (a) obtaining a biological sample from the patient; (b) assessing, determining and/or detecting the methylation status of the gene DCR1, the gene WRN, and/or regulatory regions of these genes in the biological sample: and (c) selecting capecitabine or another agent over capiri or irinotecan for the treatment if the presence of methylation (or hypermethylation) of the gene and/or their regulatory sequences is determined or detected, or if a higher level of methylation (or hypermethylation) of the gene and/or their regulatory sequences is determined or detected. The methods further may include administering the selected capecitabine treatment or the selected other agent to the patient. Other agents may include, but are not limited to, capecitabine treatment, capox treatment, and/or capox-B treatment.

In methods that include assessing, determining, and/or detecting expression of the DRC1 and/or WRN gene in the biological sample, a suitable treatment regimen for the patient may include capiri or irinotecan where DCR1 and/or WRN gene expression is detected or determined and capecitabine or another treatment over capiri or irinotecan where DCR1 and/or WRN gene expression is not detected or where a only low level of DCR1 and/or WRN gene expression is detected.

As discussed in the example section, gene methylation has a role in determining a how a patient will response to irinotecan treatment. Accordingly, the disclosed methods include treating a colon cancer patient with capecitabine, irinotecan or their combination capiri comprising: (a) obtaining a biological sample from the patient, (b) assessing, determining, and/or detecting the methylation status of a gene selected from a group consisting of DCR1, WRN, and/or regulatory regions thereof in a biological sample obtained from the patient, and (c) treating the patient with irinotecan in addition to capecitabine if the absence of methylation (or hypermethylation) of the gene and/or their regulatory sequences is determined or detected, or if a lower level of methylation (or hypermethylation) of the gene and/or their regulatory sequences is determined or detected. Preferably, the patient is selected for first-line capiri treatment.

In a related aspect, also disclosed are the uses of capecitabine, irinotecan or their combination capiri in treating cancer in a patient, wherein the patient has been selected for treatment on the basis of the methods disclosed herein. For example, capecitabine, irinotecan or their combination capiri may be used for treating a patient where the methylation status of DCR1, WRN, and/or their regulatory regions has been assessed in a biological sample from the patient as discussed herein. Further, capecitabine, irinotecan or their combination capiri may be used for treating a patient where the expression of DCR1 and/or WRN has been assessed in a biological sample from the patient as discussed herein.

Accuracy and sensitivity of the presently disclosed methods may be achieved by using a combination of markers. Any combination of markers for detecting a specific cancer, for treating a cancer, or selecting a suitable course of treatment or a suitable patient for treatment may be used, and comprises the identified markers. These may be combined with other markers known in the art. Each of the combinations for two, three four, five, or more markers, for example, can be readily and specifically envisioned given the specific disclosures of the individual marker provided herein.

As shown in the example section, the presently disclosed methods may utilize techniques for measuring the methylation status of certain genes. Various techniques for assessing methylation status of a gene are known in the art and can be utilized in the presently disclosed methods: sequencing, methylation-specific PCR (MS-PCR), melting curve methylation-specific PCR (McMS-PCR), MLPA with or without bisulphite treatment, QAMA (Zeschnigk et al, 2004), MSRE-PCR (Melnikov et al, 2005), MethyLight (Eads, C. A., Danenberg, K. D., Kawakami, K, Saltz, L. B., Blake C., shibata, D; Danenberg, P. V. and Laird P. W. Nucleic acid Res. 2000, 28: E32), ConLight-MSP (Rand K., Qu, W., Ho. T., Clark, S. J., Molloy, P. Methods. 2002, 27:114-120), bisulphite conversion-specific methylation-specific PCR (BS-MSP) (Sasaki, M., Anast, J., Bassett, W., Kawakami, T., Sakuragi, N., and Dahiya, R. Biochem. Biophys. Res. Commun. 2003, 209: 305-309), COBRA (which relies upon use of restriction enzymes to reveal methylation dependent sequence differences in PCR products of sodium bisulphite—treated DNA), methylation-sensitive single-nucleotide primer extension conformation (MS-SNuPE), methylation-sensitive single-strand conformation analysis (MS-SSCA), Melting curve combined bisulphite restriction analysis (McCOBRA)(Akey, D. T., Akey, J. M., Zhang, K., Jin, L., 2002. Genomics, 80:376-384.), PyroMethA, HeavyMethyl (Cottrell, S., Distler, J., Goodman, N., Mooney, S., Kluth, A., Olek, A., Schwope, I., Tetzner. R., Ziebarth, H., Berlin, K. Nucleic Acid Res. 2004, 32:E10), MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques for DNA methylation analysis is provided in Nucleic acids research, 1998, Vol. 26, No. 10, 2255-2264, Nature Reviews, 2003, Vol. 3, 253-266; Oral Oncology, 2006, Vol. 42, 5-13, which references are incorporated herein in their entirety.

The methylation status of a nucleic acid encoding an enzyme can be determined by any method known in the art. Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I.

Alternatively, chemical reagents can be used which selectively modify either the methylated or non-methylated form of CpG dinucleotide motifs. Suitable chemical reagents include hydrazine and bisulphite ions, and preferably bisulphite ions. The bisulphite conversion relies on treatment of DNA samples with sodium bisulphite which converts unmethylated cytosine to uracil, while methylated cytosines are maintained (Furuichi et al., 1970). This conversion finally results in a change in the sequence of the original DNA. It is general knowledge that the resulting uracil has the base pairing behaviour of thymidine which differs from cytosine base pairing behaviour. This makes the discrimination between methylated and non-methylated cytosines possible. Useful conventional techniques of molecular biology and nucleic acid chemistry for assessing sequence differences are well known in the art and explained in the literature. See, for example. Sambrook, J., et al., Molecular cloning: A laboratory Manual, (2001) 3^{rd} edition, Cold Spring Harbor, N.Y.: Gait, M. J. (ed.), Oligonucleotide Synthesis, A Practical Approach, IRL Press (1984); Hames B. D., and Higgins, S. J. (eds.). Nucleic Acid Hybridization, A Practical Approach, IRL Press (1985); and the series, Methods in Enzymology, Academic Press, Inc.

In a preferred embodiment, the methylation status of the at least one gene selected from WRN and DCR1 is determined using methylation specific PCR (MSP), or an equivalent amplification technique. In the MSP approach, DNA may be amplified using primer pairs designed to distinguish methylated from unmethylated DNA by taking advantage of sequence differences as a result of sodium-bisulphite treatment (Herman J O, Graff J R, Myohanen S, Nelkin B D, Baylin S B. Proc. Natl. Acad. Sci. USA. 1996: 93(18):9821-9826; and WO 97/46705). After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR).

The amplification products can be optionally hybridized to specific oligonucleotide probes which may also be specific for certain products. Such probes can be hybridized directly to modified DNA or to amplification products of modified DNA. Alternatively, oligonucleotide probes can be used which will hybridize to amplification products from both modified and nonmodified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Oligonucleotide primers and/or primer pairs also are disclosed herein, for example, oligonucleotide primers and/or primer pairs that specifically hybridize under amplification conditions to a gene selected from the group consisting of WRN and DCR1. Preferably, the primer and/or primer pair are designed to detect the methylation status of the gene and will specifically hybridize to the sequence of a methylated DNA following treatment with a reagent. In one particular embodiment, primers useful in MSP carried out on the gene selected from WRN and DCR1 are provided. These primers and amplicons comprise, consist essentially of or consist of the sequences listed in Table 6.

Variants of these sequences may be utilized in the presently disclosed methods. In particular, additional flanking sequences may be added, for example to improve binding specificity, as required. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences of the primers and/or probes set forth herein. The primers and probes may incorporate synthetic nucleotide analogues as appropriate or may be DNA, RNA or PNA based for example, or mixtures thereof. Similarly alternative fluorescent donor and acceptor moieties/FRET pairs may be utilized as appropriate. In addition to being labeled with the fluorescent donor and acceptor moieties, the primers and probes may include modified oligonucleotides and other appending groups and labels provided that the functionality as a primer and/or probe in the disclosed methods is not compromised.

Real-time quantitative MSP (QMSP) permits reliable quantification of methylated DNA in real time. Real-time methods are generally based on the continuous optical monitoring of an amplification procedure and utilize fluorescently labeled reagents whose incorporation in a product can be quantified and whose quantification is indicative of copy number of that sequence in the template. One such reagent is a fluorescent dye, called SYBR Green I that preferentially binds double-stranded DNA and whose fluorescence is greatly enhanced by binding of double-stranded DNA. Alternatively, labeled primers and/or labeled probes can be used for quantification. They represent a specific application of the well-known and commercially available real-time amplification techniques such as TAQMAN®, MOLECULAR BEACONS®, AMPLIFLUOR® and SCORPION® DzyNA®, Plexor™ etc. In the real-time PCR system, it is possible to monitor the PCR reaction during the exponential phase where the first significant increase in the amount of PCR product correlates to the initial amount of target template.

Accordingly, in a preferred embodiment, the methylation status of the gene of interest is determined by methylation specific PCR, preferably real-time methylation specific PCR (QMSP). In specific embodiments, the real-time methylation specific PCR comprises use of TAQMAN® probes and/or MOLECULAR BEACONS® probes and/or AMPLIFLUOR® primers and/or FRET probes and/or SCORPION® primers and/or oligonucleotide blockers and/or DzyNA® primers.

Alternatively, the methylation status of the gene of interest, is determined by methylation specific PCR amplification and, preferably the methylation specific PCR is monitored at the end-point of the amplification. Many applications do not require quantification and Real-Time PCR is used only as a tool to get convenient results presentation and storage, and at the same time to avoid post-PCR handling. Thus, analyses can be performed only to confirm whether the target DNA is present in the sample or not. Such end-point verification is carried out after the amplification reaction has finished. This knowledge can be used in a medical diagnostic laboratory to detect a predisposition to, or the incidence of, cancer in a patient. End-point PCR fluorescence detection techniques can use the same approaches as widely used for Real Time PCR. For example, <<Gene>> detector allows the measurement of fluorescence directly in PCR tubes.

TaqMan® technology uses linear, hydrolytic oligonucleotide probes that contain a fluorescent dye and a quenching dye. When irradiated, the excited fluorescent dye transfers energy to the nearby quenching dye molecule rather than fluorescencing (FRET principle). TaqMan® probes anneal to an internal region of the PCR product and are cleaved by the exonuclease activity of the polymerase when it replicates a template. This ends the activity of the quencher, and the reporter dye starts to emit fluorescence which increases in each cycle proportional to the rate of probe cleavage.

Molecular Beacons® probes also contain fluorescent and quenching dyes, but they are designed to adopt a hairpin structure while free in solution to bring both dyes in close proximity for FRET to occur. When the beacon hybridizes to the target during the annealing step, both dyes (donor and acceptor/quencher) are separated and an increase in fluorescence correlates with the amount of PCR product available. The experiments described herein show that Molecular Beacons® probes are particularly useful for monitoring the amplification/PCR reaction during the exponential phase. Thus, Molecular Beacons® probes may advantageously be employed in the presently disclosed methods.

With SCORPION® primers, sequence-specific priming and PCR product detection is achieved using a single oligonucleotide. The scorpion probe maintains a stem-loop configuration in the unhybridized state and FRET occurs. The 3′ portion of the stem also contains a sequence that is complementary to the extension product of the primer. This sequence is linked to the 5′ end of a specific primer via a non-amplifiable monomer. After extension of the SCORPION® primers, the specific probe sequence is able to bind to its complement within the extended amplicon, thus opening up the hairpin loop and providing a fluorescence signal.

In similar fashion to SCORPION® primers, the Amplifluor® technique relies upon incorporation of a Molecular Beacon® type probe into a primer. Again, the hairpin structure of the probe forms part of an amplification primer itself. However, in contrast to Scorpions® type primers, there is no block at the 5′ end of the probe in order to prevent it being amplified and forming part of an amplification product. Accordingly, the primer binds to a template strand and directs synthesis of the complementary strand. The primer therefore becomes part of the amplification product in the first round of amplification. When the complimentary strand is synthesised amplification occurs through the hairpin structure. This separates the fluorophore and quencher molecules, thus leading to generation of fluorescence as amplification proceeds.

In a variant Amplifluor® format, the sequence-specific primer carries a “Z” sequence addition at its 5′ end and yields an initial amplification product that contains the complement of the “Z” sequence. A second primer with stem-loop configuration is designed to contain the “Z” sequence and anneals to the template containing the complement of “Z”. During the polymerization reaction the reporter and quencher molecules are incorporated into the product. This product serves as a template for further amplification. As the hairpin conformation of the template becomes unfolded during polymerization, a fluorescence signal is observed.

In the Heavymethyl® technique, the priming is methylation specific, but non-extendable oligonucleotide blockers provide this specificity instead of the primers themselves. The blockers bind to bisulphite-treated DNA in a methylation-specific manner, and their binding sites overlap the primer binding sites. When the blocker is bound, the primer cannot bind and therefore the amplicon is not generated. The Heavymethyl® technique can be used in combination with real-time or end point detection.

The Plexor™ qPCR and qRT-PCR Systems take advantage of the specific interaction between two modified nucleotides to achieve quantitative PCR analysis. One of the PCR primers contains a fluorescent label adjacent to an iso-dC residue at the 5′ terminus. The second PCR primer is unlabeled. The reaction mix includes deoxynucleotides and iso-dGTP modified with the quencher dabcyl. Dabcyl-iso-dGTP is preferentially incorporated at the position complementary to the iso-dC residue. The incorporation of the dabcyl-iso-dGTP at this position results in quenching of the fluorescent dye on the complementary strand and a reduction in fluorescence, which allows quantitation during amplification. For these multiplex reactions, a primer pair with a different fluorophore is used for each target sequence.

In real-time embodiments, quantitation may be on an absolute basis, or may be relative to a constitutively methylated DNA standard, or may be relative to an unmethylated DNA standard. Methylation status may be determined by using the ratio between the signal of the marker under investigation and the signal of a reference gene where methylation status is known (such as β-actin for example), or by using the ratio between the methylated marker and the sum of the methylated and the non-methylated marker. Alternatively, absolute copy number of the methylated marker gene can be determined.

Suitable controls may need to be incorporated in order to ensure the method chosen is working correctly and reliably. Suitable controls may include assessing the methylation status of a gene known to be methylated. This experiment acts as a positive control to ensure that false negative results are not obtained. The gene may be one which is known to be methylated in the sample under investigation or it may have been artificially methylated. In one embodiment, the gene of interest may be assessed in normal cells, following treatment with SssI methyltransferase, as a positive control. Additionally or alternatively, suitable negative controls may be employed in the disclosed methods. Here, suitable controls may include assessing the methylation status of a gene known to be unmethylated or a gene that has been artificially demethylated. This experiment acts as a negative control to ensure that false positive results are not obtained. In one embodiment, the gene of interest may be assessed in normal cells as a negative control, in particular if the gene is unmethylated in normal tissues.

Other techniques for assessing methylation in a test sample comprise sequencing. Epigenomic variation, as an extension of genome sequencing applications, can be investigated using next-generation sequencing approaches that enable the ascertainment of genome-wide patterns of methylation and how these patterns change in the context of disease, and under various other influences such as treatment of disease with certain agents. Next Generation Sequencing (NGS) is a term well known in the art that has come to mean post-Sanger sequencing methods.

Also disclosed herein are kits for assessing methylation in a test sample. The kit comprises optionally a reagent that (a) modifies methylated cytosine residues but not non-methylated cytosine residues, or that (b); modifies non-methylated cytosine residues but not methylated cytosine residues. The kit also comprises a pair of oligonucleotide primers that specifically hybridizes under amplification conditions to the methylated gene following treatment with a reagent, which gene is selected from the group consisting of WRN and/or DCR1.

Kits, as contemplated herein, are assemblages of reagents that be utilized for testing methylation. They are typically in a package which contains all elements, optionally including instructions. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentially modifying methylated and non-methylated cytosine residues. Typically the kit will contain oligonucleotide primers which specifically hybridize to regions within 1 kb of the transcription start sites of the genes identified in Table 2. Typically the kit will contain both a forward and a reverse primer for a single gene. If there is a sufficient region of complementarity, e.g., 12, 15, 18, or 20 nucleotides, then the primer may also contain additional nucleotide residues or other chemical moieties that do not interfere with hybridization but may be useful for other manipulations. Exemplary of such other residues may be sites for restriction endonuclease cleavage, for ligand binding or for factor binding or linkers. Other moieties may include detectable labels or specific binding moieties, such as biotin. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase and deoxyribonucleotides. Means of detection may also be provided in the kit, including detectable labels on primers or probes. Kits may also contain reagents for detecting gene expression for one of the markers (e.g., DCR1 and/or WRN). Such reagents may include probes, primers, or antibodies, for example. In the case of enzymes or ligands, substrates or binding partners may be used to assess the presence of the marker.

Also provided is a method of diagnosing or prognosing cancer comprising determining the methylation status of the gene of interest in a sample obtained from a patient, wherein the methylation status is assessed using the methods disclosed herein. In one embodiment, methylation (or hypermethylation) of DCR1, WRN, and/or their regulatory regions may indicate that cancer is present or that irinotecan-resistant CRC is present. The reverse situation is also applicable and nonmethylation (or hypomethylation) of DCR1, WRN, and/or their regulatory regions may indicate that cancer is not present, that irinotecan-resistant CRC is not present, and/or that irinotecan-sensitive CRC is present.

### EXAMPLES

The following examples are illustrative and are not intended to limit the scope of the present invention.

Predictive and Prognostic Methylation Markers for the Outcome after Treatment of CRC

### Materials and Methods

### Candidate Gene Selection

Drug activity data sets are publicly available from a number of sources. Here, methylation data for a number of DNA markers was correlated to drug activity data provided by The Genomics and Bioinformatics Group, 2000 Publications Data Set, Drug Activity of 118—Mechanism of Action Drugs, available at its website.

To generate the methylation data, 1156 assays were tested against 32 cell lines from breast cancer (BT549, HSS78T, MCF7, MDAMB231, T47D), colon cancer (Colo205, HCT116, HCT15, HT29, SW620), lung cancer (A549, H226, H23, H460, H522), leukemia (CCRF-CEM, HL60, K563, MOLT4, RPMI8226, SR), melanoma (MALME3M, SK-MEL2, SK-MEL5, SK-MEL28), ovarian cancer (OVCAR3, SKOV3), prostate cancer (DUI45, PC3) and renal cancer (7860, A498). The 1156 assays were designed to cover the TSS proximal CpG island of 631 genes involved in DDR (DNA Damage Repair and Response). Of the 1156 assays tested, 562 assays (389 genes) were retained for which we observed at least one methylated and one unmethylated cell line sample. For the same set of 32 cell lines the −log(GI50) scores of 118 drugs from the NCI60 database were selected. These drugs were grouped into 15 common mode of actions (MOA's).

The above datasets were combined to correlate the methylation profile of 562 assays to the activity profile of 118 drugs and 15 MOA's. For each of the 562×118 couples (assay,drug) and the 562×15 couples (assay,MOA) a p-value was computed via randomization. Given a couple (assay,drug) or (assay,MOA), the methylation profile of the assay was used as a starting point for the randomization experiment. This profile divides the set of cell lines in methylated and unmethylated ones (cell lines where the methylation call is missing were ignored). For both subsets of cell lines the average−log(GI50) score of the drug (or MOA): avgM(−log(GI50)) and avgU(−log(GI50)) was computed. The larger the difference between both averages, the more predictive the assay is of sensitivity to the drug (or MOA). If avgM>avgU it was assumed that methylation indicated higher sensitivity and the difference as avgM-avgU was computed. Otherwise we assumed the unmethylated state indicated higher sensitivity and computed the difference as avgU−avgM.

Using difference avgM−avgU or avgU−avgM as a reference, a randomization experiment consisting of 10 million iterations was conducted. In each iteration a stratified sample from the 32 cell lines was selected, the difference between the average−log(GI50) in selected and unselected cell lines was computed, and it was 30 counted how often this difference was at least as high as the reference difference, and the result was divided by 10 million to obtain a p-value. The stratified sampling strategy was based on the categorization of the 32 cell lines into 8 subtypes: breast (5), colon (5), leukemia (6), lung (5), melanoma (4), ovarian (2), prostate (2) and renal (3). To compose a random sample, we randomly selected within each subtype the number of methylated (in case avgM>avgU) or unmethylated (in case avgU>avgM) cell lines within that subtype. This was done to favor markers that discriminate between high and low sensitivity within different tissue types.

Robust assays were identified and selected. Those assays are highly predictive for the response of cell lines to single drug or to a group of drugs with a common mode of action. The mode of action taken into consideration for the present study was topoisomerase I.

Quality control was performed using in vitro methylated DNA sample, unmethylated DNA sample and no template control sample (H2O). From the Lightcycler platform, the cycle threshold (ct) and melting temperature (Tm) calling are calculated by the Roche Lightcycler 480 software (Software release 1.5.0). From the capillary electrophoresis platform, the band sizes and band heights are calculated by the Caliper software (Caliper Labchip HT version 2.5.0, Build 195 Service Pack 2).

In a first stage, the melting temperature and product size of in vitro methylated DNA are measured for a marker. A sample is called positive for that marker if the melting temperature and product size are within the specified boundaries of a measured in vitro methylated reference. Additional rules are imposed on the Ct value and the band intensity of the product with the right size. Product size has to be within the reference product size+/−10 bp interval. Melting temperature has to be within the reference product temperature+/−2 degrees Celsius range. In addition, the cycle threshold has to be under 40 cycles and the correct band intensity height has to be higher than 20, the latter is a relative number calculated by the caliper software.

### Methylation Analysis of Cell Lines

Cell lines were purchased at ATCC or ECCAC and cultured under the prescribed conditions in the certificate of analysis. HCT15, HCT116, LS513, LS174T, Colo320, SW48, SW1398, HT29, Colo205, SW480, and RKO were cultured in Dulbecco's modified Eagle's medium (DMEM; Lonza Biowhittaker, Verviers, Belgium) containing 10% fetal bovine serum (Hyclone, Perbio, UK). Caco-2 was cultured in RPMI 1640 (Lonza Biowhittaker) containing 20% fetal bovine serum. LIM 1863 was cultured in RPMI 1640 (Lonza Biowhittaker) containing 5% FCS, 0.01 mg/ml thioglycerol, 1 mg/ml insulin and 1 μg/ml hydrocortisone. All cell culture media were supplemented with 2 mM L-glutamine, 100 IU/ml sodium penicillin (Astellas Pharma B.V., Leiderdorp. The Netherlands) and 100 mg/ml streptomycin (Fisiopharma, Palomonta (SA), Italy). To investigate re-expression of DCR1 after inhibition of DNA metyltransferases, HCT116 cells were treated with 5000 nM 5-aza-2′-deoxycytidine for 3 days (DAC, Sigma Chemical Co., St. Louis. Mo., USA).

DNA was manually macrodissected from areas containing >70% tumor cell content and isolated by a column-based method (Qlamp DNA microkit, Qiagen. Hilden, Germany) as described before (Brosens R P et al., J Pathol 2010; 221:411-24; Buffan T E et al., Cell Oncol 2007; 29:351-9.). DNA concentrations were quantified using the Nanodrop 1000 UV spectrophotometer (Nanodrop Technologies Inc, Wilmington, Del. USA). DNA was subjected to sodium bisulfite conversion using the EZ DNA Methylation Kit (Zymo Research, Orange, Calif., USA) according to the manufacturer's protocol.

The discovery set was subjected to high-throughput lightcycler MSP assay for the 23 selected candidate genes. Per sample, 20 ng bisulfite-modified DNA was amplified with methylation specific primer sets with the following PCR conditions: 95° C. for 10 minutes followed by 45 cycles of 95° C. for 10 seconds, 60° C. for 30 seconds and 72° C. for 1 second. The kit used to amplity was the LightCycler 480 SYBR Green I Master kit (Roche, Vilvoorde. Belgium). The amplicons were checked for size and quantified by capillary electrophoresis (LC90 Labchip; Caliper Lifesciences). Quality control (QC) was performed with bisulfite converted in vitro Methylated DNA and bisulfite converted HCT116 DKO DNA. In vitro Methylated DNA is commercial available (Chemicon, Temecula, Calif.) and served as a positive control. As a negative control, DNA from the Human HCT116 DKO cell line was used. These cells contain genetic knockouts of both DNA methyltransferases DNMT1 (−/−) and DNMT3b (−/−). The DNA derived from HCT116 DKO cells has a low level of DNA methylation (<5%). Amplification of beta-actin was used as an unmethylated reference gene.

CRC cell lines and the CAIRO validation set were subjected to a quantitative MSP assay for DCR1. Per sample, bisulfite-modified DNA was used to amplify with unmethylated or methylated DNA specific primer sets. qMSP reactions were carried out in a 25 μl reaction volume containing 36 ng of bisulfite-treated DNA. 10 pmol of each primer and 1× Power SYBR Green PCR Master Mix (Applied Biosystems, Foster City, Calif.). Each plate included no template controls and a standard curve with a serial dilution of bisultite-modified DNA from a mixture of methylated cell line (HCT116) and unmethylated cell line (HCT116 DKO). Thermocycling parameters were 95° C. for 15 minutes, followed by 40 cycles at 95° C. for 30 seconds, 56° C. for 30 seconds and 72° C. for 30 seconds. Amplicons were checked for size using a melting curve. Melting cycle parameters were 95° C. for 15 seconds, 60° C. for 60 seconds and 95° C. for 15 seconds. All samples were run and analyzed in duplicate. Cycle threshold (Ct) values were measured at a fixed fluorescence threshold (i.e., 0.01), which was always in the exponential phase of the amplification curves. The methylation percentage per sample was calculated according to the formula 2e−[mean Ct M reaction)/(2e−[mean Ct M reaction]+2e−mean Ct U reaction])*00. The U (unmethylated) and M (methylated) reactions were amplified with comparable efficiencies. Methylation outcomes were dichotomized (positive versus negative) using as a cut-off point the highest methylation percentage (4%) as measured three times in duplicates in 21 normal colon mucosa's from non-cancer patients. Primer sequences of the DCR1 MSP assays (for U=assay for detection of unmethylated DCR1; for M=assay for detection of methylated DCR1) can be found in Table 3.

### Study Design

The study represents a retrospective case-control study on which the candidate-gene approach was applied. Tumor material was available from a subgroup of patients that participated in a randomized phase III study, the CAIRO study of the Dutch Colorectal Cancer Group (DCGG), registered with ClinicalTrials.gov with the number NCT00312000 (Koopman M et al., Lancet 2007; 370:135-42; Casparie M et al., Cell Oncol 2007; 29): 19-24).

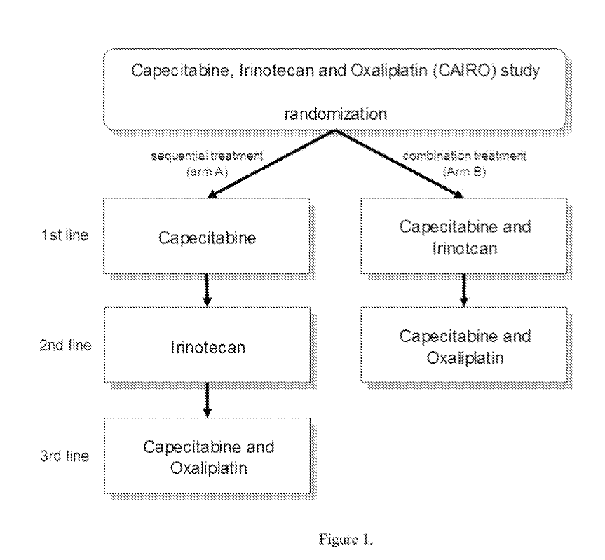

In this study, 820 patients with metastatic CRC were randomized between either sequential (arm A) or combination (arm B) treatment with capecitabine, irinotecan and oxaliplatin. Patients in Arm A received first-line capecitabine, second-line irinotecan and third-line capecitabine plus oxaliplatin (CAPOX). Patients in Arm B received first-line capecitabine plus irinotecan (CAPIRI) and second-line CAPOX (see FIG. 1). The identification of predictive markers to capecitabine, irinotecan, and/or oxaliplatin was based on progression free survival (PFS). It only included patients that received ≧3 cycli of a certain treatment-line or ≧2 cycli when cause of death was progressive disease. PFS for first-line treatment was calculated from the date of randomization to the first observation of disease progression or death from any cause reported after first-line treatment. PFS for second-line treatment was calculated from the first observation of disease progression from the first-line treatment to disease progression or death from any cause reported after second-line treatment. PFS for third-line treatment was calculated likewise.

Formalin-fixed paraffin-embedded tissue samples from primary tumors, resected before chemotherapy, from 543 patients from the CAIRO study were available for DNA isolation. For the present study, tumor DNA samples from 351 patients were used and split in a discovery set (n=185; 90 from arm A, 95 from arm B) and a validation set (n=166; 78 from arm A, 88 from arm B). For the discovery set, patients were selected based on tumor cell percentage (>70%) and stratification variables that were matched according to the stratification factors in the original study (for the subgroup of patients that underwent resection), i.e. performance status, predominant localization of metastases, previous adjuvant therapy and serum lactate dehydrogenase level (LDH). Table 1 shows the clinical characteristics of patients included in the present study and of all patients that participated in the CAIRO study. For both the discovery and the validation set, only patients that had received at least 3 cycles of therapy, or 2 cycles when cause of death was progressive disease, were included.

TABLE 1
Clinical characteristics of patients included in the present study and of all patients that participated the CAIRO Study

| | Sequential treatment (arm A) | | | | | | Combination treatment (arm B) | | | | | | Total | | | |
|---|---|---|---|---|---|---|---|---|---|---|---|---|---|---|---|---|
| | Original | | Present study | | Present study | | Original | | Present study | | Present study | | Original | | | |
| | CAIRO study | | Discovery set | | Verification set | | CAIRO study | | Discovery set | | Verification set | | CAIRO study | | Present study | |
| | (n = 401) | | (n = 90) | | (n = 78) | | (N = 402) | | (n = 95) | | (n = 88) | | (n = 803) | | (n = 351) | |
| Age | | | | | | | | | | | | | | | | |
| Age at | 64 | (27-84) | 64 | (41-82) | 63 | (36-77) | 63 | (31-81) | 61 | (36-80) | 61 | (37-38) | 63 | (27-84) | 62 | (36-82) |
| randomisation | | | | | | | | | | | | | | | | |
| (years) | | | | | | | | | | | | | | | | |
| >70 years | 93 | (23%) | 21 | (23%) | 21 | (27%) | 81 | (20%) | 19 | (20%) | 12 | (14%) | 174 | (22%) | 73 | (21%) |
| Gender | | | | | | | | | | | | | | | | |
| Male | 252 | (63%) | 55 | (61%) | 46 | (59%) | 255 | (63%) | 61 | (64%) | 58 | (66%) | 507 | (63%) | 220 | (63%) |
| Female | 149 | (37%) | 35 | (39%) | 32 | (41%) | 147 | (37%) | 34 | (36%) | 30 | (34%) | 296 | (37%) | 131 | (37%) |
| Performance | | | | | | | | | | | | | | | | |
| status | | | | | | | | | | | | | | | | |
| PS0 | 257 | (64%) | 57 | (63%) | 54 | (69%) | 244 | (61%) | 64 | (67%) | 57 | (65%) | 501 | (62%) | 232 | (66%) |
| PS1 | 128 | (31%) | 29 | (32%) | 23 | (29%) | 142 | (35%) | 27 | (28%) | 28 | (32%) | 268 | (33%) | 107 | (30%) |
| PS2 | 18 | (5%) | 4 | (4%) | 1 | (1%) | 16 | (4%) | 4 | (4%) | 3 | (3%) | 34 | (4%) | 12 | (3%) |
| Predominant | | | | | | | | | | | | | | | | |
| localisation | | | | | | | | | | | | | | | | |
| of metastases | | | | | | | | | | | | | | | | |
| Liver | 277 | (69%) | 64 | (71%) | 55 | (71%) | 285 | (71%) | 65 | (68%) | 62 | (70%) | 562 | (70%) | 246 | (70%) |
| Extrahepatic | 118 | (29%) | 24 | (27%) | 22 | (28%) | 115 | (29%) | 29 | (31%) | 25 | (28%) | 233 | (29%) | 100 | (28%) |
| Unknown | 6 | (2%) | 2 | (2%) | 1 | (1%) | 2 | (<1%) | 1 | (1%) | 1 | (1%) | 8 | (<1%) | 5 | (1%) |
| LDH at | | | | | | | | | | | | | | | | |
| randomisation | | | | | | | | | | | | | | | | |
| Normal | 256 | (64%) | 65 | (72%) | 53 | (68%) | 257 | (64%) | 70 | (74%) | 57 | (65%) | 513 | (64%) | 245 | (70%) |
| Abnormal | 145 | (36%) | 25 | (28%) | 25 | (32%) | 145 | (36%) | 25 | (26%) | 31 | (35%) | 290 | (36%) | 106 | (30%) |
| Previous | | | | | | | | | | | | | | | | |
| adjuvant | | | | | | | | | | | | | | | | |
| therapy | | | | | | | | | | | | | | | | |
| Yes | 55 | (14%) | 16 | (18%) | 13 | (17%) | 56 | (14%) | 18 | (19%) | 16 | (18%) | 111 | (14%) | 63 | (18%) |
| No | 346 | (86%) | 74 | (82%) | 65 | (83%) | 346 | (86%) | 77 | (81%) | 72 | (82%) | 692 | (86%) | 288 | (82%) |
| Site of | | | | | | | | | | | | | | | | |
| primary tumour | | | | | | | | | | | | | | | | |
| Colon | 251 | (63%) | 63 | (70%) | 69 | (76%) | 227 | (57%) | 56 | (59%) | 51 | (58%) | 478 | (80%) | 229 | (85%) |
| Rectosigmoid | 28 | (7%) | 7 | (8%) | 2 | (3%) | 32 | (8%) | 6 | (6%) | 8 | (9%) | 60 | (8%) | 23 | (7%) |
| Rectum | 119 | (30%) | 19 | (21%) | 16 | (21%) | 141 | (35%) | 32 | (34%) | 29 | (33%) | 280 | (32%) | 96 | (27%) |
| Multiple | 2 | (<1%) | 1 | (1%) | 1 | (1%) | 2 | (<1%) | 1 | (1%) | 0 | (0%) | 4 | (<1%) | 3 | (1%) |
| tumours | | | | | | | | | | | | | | | | |
| Missing | 1 | (<1%) | 0 | (0%) | 0 | (0%) | 0 | (0%) | 0 | (0%) | 0 | (0%) | 1 | (<1%) | 0 | (0%) |

TABLE 2
Correlation between methylation and outcome with respect to progression-free survival (PFS)

| | | | | | Median | | HR (methylated | | | |
|---|---|---|---|---|---|---|---|---|---|---|
| | Treatment | | Methylation | | PFS in days | | vs unmethylated) | | | Corrected |
| | arm | First-line therapy | status | nr of patients | Median PFS | 95% CI | HR | 95% CI | p-value | p-value |
| BK | A | Capecitabine | U | 66 | 188 | 131-216 | 1.1 | 0.7-1.7 | 0.8 | 0.8 |
| | | | M | 24 | 149 | 118-253 | | | | |
| | B | Capecitabine + irinotecan | U | 69 | 251 | 212-296 | 0.9 | 0.6-1.4 | 0.7 | 0.8 |
| | | | M | 26 | 253 | 218-330 | | | | |
| CAT | A | Capecitabine | U | 78 | 190 | 133-210 | 0.9 | 0.5-0.7 | 0.8 | 0.9 |
| | | | M | 12 | 116 | 67-NA | | | | |
| | B | Capecitabine + irinotecan | U | 81 | 258 | 240-298 | 0.7 | 0.4-1.3 | 0.2 | 0.6 |
| | | | M | 14 | 190 | 166-410 | | | | |
| CCND2 | A | Capecitabine | U | 63 | 156 | 126-208 | 1.3 | 0.8-2.0 | 0.3 | 0.6 |
| | | | M | 27 | 202 | 169-322 | | | | |
| | B | Capecitabine + irinotecan | U | 64 | 251 | 213-296 | 1.2 | 0.8-2.0 | 0.4 | 0.7 |
| | | | M | 31 | 253 | 197-378 | | | | |
| CDK5 | A | Capecitabine | U | 75 | 191 | 133-216 | 0.7 | 0.4-1.3 | 0.2 | 0.5 |
| | | | M | 15 | 125 | 67-246 | | | | |
| | B | Capecitabine + irinotecan | U | 78 | 253 | 217-296 | 1.3 | 0.8-2.5 | 0.3 | 0.6 |
| | | | M | 17 | 218 | 189-439 | | | | |
| DAPK1 | A | Capecitabine | U | 66 | 188 | 129-210 | 1.0 | 0.6-1.4 | 0.8 | 0.8 |
| | | | M | 24 | 144 | 125-237 | | | | |
| | B | Capecitabine + irinotecan | U | 77 | 243 | 212-262 | 1.7 | 1.0-2.5 | 0.05 | 0.5 |
| | | | M | 18 | 307 | 241-500 | | | | |
| DCR1 | A | Capecitabine | U | 48 | 178 | 127-202 | 1.4 | 0.9-2.0 | 0.1 | 0.5 |
| | | | M | 42 | 184 | 128-278 | | | | |
| | B | Capecitabine + irinotecan | U | 65 | 270 | 246-303 | 0.4 | 0.3-0.7 | 0.0009 | 0.02 |
| | | | M | 30 | 191 | 162-258 | | | | |
| EEF1A2 (1 | A | Capecitabine | U | 71 | 168 | 127-208 | 1.2 | 0.7-2.0 | 0.5 | 0.7 |
| | | | M | 19 | 202 | 125-340 | | | | |
| | B | Capecitabine + irinotecan | U | 75 | 260 | 228-301 | 0.7 | 0.4-1.3 | 0.2 | 0.5 |
| | | | M | 20 | 212 | 174-351 | | | | |
| EEF1A2 (2 | A | Capecitabine | U | 83 | 177 | 129-202 | 1.3 | 0.6-2.5 | 0.5 | 0.7 |
| | | | M | 7 | 246 | 77-NA | | | | |
| | B | Capecitabine + irinotecan | U | 93 | 251 | 217-286 | 2.3 | 0.6-10.0 | 0.2 | 0.5 |
| | | | M | 2 | 478 | 378-NA | | | | |
| HOXA9 | A | Capecitabine | U | 51 | 191 | 133-234 | 0.8 | 0.6-1.3 | 0.4 | 0.7 |
| | | | M | 39 | 168 | 124-216 | | | | |
| | B | Capecitabine + irinotecan | U | 60 | 248 | 213-296 | 1.0 | 0.7-1.7 | 0.9 | 0.9 |
| | | | M | 35 | 260 | 212-302 | | | | |
| IRAK1 | A | Capecitabine | U | 56 | 130 | 119-199 | 1.6 | 1.0-2.5 | 0.03 | 0.3 |
| | | | M | 33 | 216 | 159-321 | | | | |
| | B | Capecitabine + irinotecan | U | 55 | 258 | 218-296 | 0.7 | 0.4-1.0 | 0.07 | 0.5 |
| | | | M | 40 | 236 | 191-301 | | | | |
| LIG4 | A | Capecitabine | U | 7 | 190 | 65-NA | 1.2 | 0.6-2.5 | 0.6 | 0.8 |
| | | | M | 82 | 173 | 127-210 | | | | |
| | B | Capecitabine + irinotecan | U | 8 | 848 | 301-NA | 0.5 | 0.2-1.0 | 0.03 | 0.4 |
| | | | M | 86 | 246 | 213-272 | | | | |
| NUDT1 | A | Capecitabine | U | 4 | 99 | 60-NA | 1.5 | 0.6-5.0 | 0.4 | 0.7 |
| | | | M | 85 | 187 | 129-208 | | | | |
| | B | Capecitabine + irinotecan | U | 10 | 298 | 191-NA | 0.6 | 0.3-1.3 | 0.1 | 0.5 |
| | | | M | 84 | 251 | 213-286 | | | | |
| PAX3 (1) | A | Capecitabine | U | 14 | 126 | 65-475 | 1.2 | 0.7-2.0 | 0.6 | 0.8 |
| | | | M | 76 | 189 | 133-210 | | | | |
| | B | Capecitabine + irinotecan | U | 12 | 284 | 228-NA | 0.8 | 0.4-1.4 | 0.5 | 0.7 |
| | | | M | 83 | 248 | 213-288 | | | | |
| PAX3 (2) | A | Capecitabine | U | 9 | 127 | 60-NA | 1.3 | 0.6-2.5 | 0.5 | 0.7 |
| | | | M | 81 | 187 | 131-216 | | | | |
| | B | Capecitabine + irinotecan | U | 11 | 301 | 218-NA | 0.6 | 0.3-1.3 | 0.2 | 0.5 |
| | | | M | 84 | 251 | 212 286 | | | | |
| PRKCB1 | A | Capecitabine | U | 67 | 168 | 127-307 | 1.1 | 0.7-1.7 | 0.7 | 0.8 |
| | | | M | 23 | 192 | 127-307 | | | | |
| | B | Capecitabine + irinotecan | U | 75 | 258 | 228-301 | 0.6 | 0.4-1.0 | 0.09 | 0.6 |
| | | | M | 20 | 213 | 181-301 | | | | |
| PROK2 | A | Capecitabine | U | 72 | 179 | 127-223 | 0.8 | 0.5-1.4 | 0.4 | 0.7 |
| | | | M | 18 | 182 | 127-246 | | | | |
| | B | Capecitabine + irinotecan | U | 77 | 251 | 212-296 | 0.6 | 0.4-1.1 | 0.1 | 0.5 |
| | | | M | 18 | 253 | 218-302 | | | | |
| PROP1 | A | Capecitabine | U | 6 | 99 | 60-NA | 2.0 | 0.8-5.0 | 0.1 | 0.5 |
| | | | M | 84 | 188 | 129-216 | | | | |
| | B | Capecitabine + irinotecan | U | 7 | 301 | 182-NA | 0.5 | 0.2-1.3 | 0.09 | 0.5 |
| | | | M | 88 | 251 | 217-272 | | | | |
| PTGS2 | A | Capecitabine | U | 81 | 168 | 127-210 | 1.1 | 0.6-2.0 | 0.8 | 0.9 |
| | | | M | 9 | 202 | 177-NA | | | | |
| | B | Capecitabine + irinotecan | U | 90 | 256 | 228-296 | 0.6 | 0.3-1.7 | 0.3 | 0.7 |
| | | | M | 5 | 181 | 162-NA | | | | |
| RASSF1 | A | Capecitabine | U | 74 | 164 | 127-202 | 1.2 | 0.7-2.0 | 0.5 | 0.7 |
| | | | M | 16 | 209 | 118-339 | | | | |
| | B | Capecitabine + irinotecan | U | 85 | 258 | 228-396 | 0.5 | 0.3-1.1 | 0.09 | 0.5 |
| | | | M | 10 | 201 | 127-NA | | | | |
| RBBP8 | A | Capecitabine | U | 55 | 177 | 131-208 | 0.9 | 0.6-1.4 | 0.7 | 0.8 |
| | | | M | 35 | 191 | 124-246 | | | | |
| | B | Capecitabine + irinotecan | U | 47 | 262 | 240-301 | 0.7 | 0.5-1.1 | 0.1 | 0.5 |
| | | | M | 48 | 218 | 200-301 | | | | |
| RHOB | A | Capecitabine | U | 86 | 188 | 129-210 | 0.7 | 0.3-2.0 | 0.5 | 0.7 |
| | | | M | 4 | 112 | 57-NA | | | | |
| | B | Capecitabine + irinotecan | U | 91 | 254 | 218-296 | 0.8 | 0.3-2.0 | 0.6 | 0.8 |
| | | | M | 4 | 206 | 134-NA | | | | |
| SPO11 | A | Capecitabine | U | 5 | 190 | 127-NA | 1.4 | 0.6-3.3 | 0.5 | 0.7 |
| | | | M | 85 | 177 | 129-208 | | | | |
| | B | Capecitabine + irinotecan | U | 2 | 200 | 191-NA | 2.5 | 0.6-10.0 | 0.3 | 0.6 |
| | | | M | 92 | 253 | 216-204 | | | | |
| TBX5 | A | Capecitabine | U | 4 | 65 | 53-NA | 2.3 | 0.8-5.0 | 0.1 | 0.5 |
| | | | M | 86 | 188 | 131-210 | | | | |
| | B | Capecitabine + irinotecan | U | 3 | 197 | 131-NA | 2.0 | 0.6-5.0 | 0.3 | 0.6 |
| | | | M | 91 | 252 | 218-296 | | | | |
| TIPARP | A | Capecitabine | U | 75 | 187 | 127-208 | 0.9 | 0.5-1.7 | 0.7 | 0.8 |
| | | | M | 15 | 177 | 65-321 | | | | |
| | B | Capecitabine + irinotecan | U | 75 | 258 | 228-296 | 0.8 | 0.5-1.4 | 0.4 | 0.7 |
| | | | M | 20 | 198 | 137-311 | | | | |

Abbreviations:
BIK = BCL2-interacting killer (apoptosis-inducing);
CAT = Catalase;
CCND2 = cyclin D2;
CDK5 = cyclin-dependent kinase 5;
DAPK1 = death-associated protein kinase 1;
DCR1 = decoy receptor 1;
EEF1A2 = eukaryotic translation elongation factor 1 alpha 2;
HOXA9 = homeobox A9;
IRAK1 = interleukin-1 receptor-associated kinase 1;
LIG4 = ligase IV.
DNA, ATP-dependent;
NUDT1 a nudix (nucleoside diphosphate linked moiety X)-type motif 1;
PAX3 = paired box 3;
PRKCB1 = protein kinase C, beta;
PROK2 = prokineticin 2;
PROP1 = PROP paired-like homeobox 1;
PTGS2 = prostaglandin-endoperoxide synthase 2 (prostaglandin G/H synthase and cyclooxygenase);
RASSF1 = Ras association (RalGDS/AF-6) domain family member 1;
RBBP8 = retinoblastoma binding protein 8;
RHOB = ras homolog gene family, member B;
SPO11 = SPO11 meiotic protein covalently bound to DSB homolog (S. cerevisiae);
TBX5 = T-box 5;
TIPARP = TCDD-inducible poly (ADP-ribose) polymerase;
U = Unmethylated;
M = Methylated;
HR = Hazard Ratio.

TABLE 3
Overview of the gene identification, assays,
forward primer sequences and reverse primer
sequences used for amplification, the converted
sequences and unconverted sequences of the
amplicons, HG19 genome version start and end
position of the amplicon.

| Gene assay | Forward | Reverse | Bisulphite | | | | |
|---|---|---|---|---|---|---|---|
| and | Primer | Primer | treated | Genomic | HG19 | HG19 | Amplicon |
| Chromosome | (5′-3′) | (5′-3′) | amplicon | sequence | start | end | Size |
| DCR1 | TTACGCG | CATCAAA | TTACGCGTACGAAT | CCACGCGCACGAAC | 22960457 | 22960584 | 127 |
| Chromosome | TACGAAT | CGACCGA | TTAGTTAACGATTT | TCAGCCAACGATTT | | | |
| 8 | TTAGTTA | AACG | TTGATAGATTTTTG | CTGATAGATTTTTG | | | |
| | AC | (SEQ ID | GGAGTTTGATTAGA | GGAGTTTGACCAGA | | | |
| | (SEQ ID | NO: 6) | GATGTAAGGGGTGA | GATGCAAGGGGTGA | | | |
| | NO: 5) | | AGGAGCGTTTTTTA | AGGAGCGCTTCCTA | | | |
| | | | TCGTTAGGGAATTT | CCGTTAGGGAACTC | | | |
| | | | TGGGGATAGAGCGT | TGGGGACAGAGCGC | | | |
| | | | TTCGGTCGTTTGAT | CCCGGCCGCCTGAT | | | |
| | | | G | G | | | |
| | | | (SEQ ID NO: 7) | (SEQ ID NO: 8) | | | |

RNA Isolation and qRT-PCR

Total RNA was isolated using TriZoI reagent (Invitrogen, Breda, The Netherlands), and subjected to purification using RNeasy Mini Kit (Qiagen). After DNAse treatment (RQI DNAse, Promega, Leiden, The Netherlands). cDNA was made with the Iscript cDNA Synthesis Kit (BioRad, Veenendaal, The Netherlands). Quantitative RT-PCR was done using TaqMan® Gene Expression Assays from Applied Biosystems directed to DCR1 (Hs00182570_m1) and B2M (Hs00984230_m1). Relative expression levels were determined by calculating the Ct-ratio (Ct ratio=2̂−(Ct DCR1−Ct B2M)).

### Statistical Analysis

The primary endpoint of the present study was progression free survival (PFS) under first-line systemic therapy with or without irinotecan stratified for methylation status of candidate genes. PFS for first-line treatment was calculated from the date of randomization to the first observation of disease progression or death reported after first-line treatment. The predictive value of candidate methylation genes for the outcome of combined irinotecan and capecitabine (capiri) compared to capecitabine alone was assessed by survival analysis including Kaplan-Meier curves. Cox Proportional Hazard models were used to estimate Hazard Ratios (HR) and 95% confidence intervals (95% CI) for methylation status per treatment, or for treatment stratified by methylation status. The statistically significant markers and clinicopathological parameters were further examined in a multivariate Cox regression model. Independence between the markers and the other covariates was analyzed by the Fisher's exact test for the discrete variables and by Spearman Ranked Correlation for age. Results were considered significant when p-values corrected for multiple testing by Benjamini and Hochberg False Discovery Rate were ≦0.05 (Benjamini Y et al., Journal of the Royal Statistical Society, 1995; Series B (Methodological):289-300). Student's T-test was used for comparison of DCR1 expression levels before and after DAC and TSA treatment of HCT116. Pearson correlation analysis was used to measure correlation between DCR1 methylation and mRNA expression levels from 78 primary CRC tissue samples as provided by The Cancer Genome Atlas (TCGA) database ([http://cancergenome.nih.gov).

### Results

### Candidate Genes

Candidate gene selection yielded 22 genes associated with the topoisomerase-I related mode of action. These genes were analyzed for DNA methylation status in the discovery set. Of 17 genes, promoter hypermethylation had not been described in CRC before. Although WRN methylation has been described as a predictive marker for response to irinotecan before and was included in our initial selection, it did not meet the criteria to be in the final selection of candidate genes in the present study.

Methylation frequencies observed in the present study for all 22 genes selected, as well as methylation frequencies in CRC from literature as far as available are shown in supplementary table 2. Methylation frequencies ranged from 5% to 98%, average 43%.

Patients with Methylated DCR1 do not Benefit from Irinotecan Added to Capecitabine

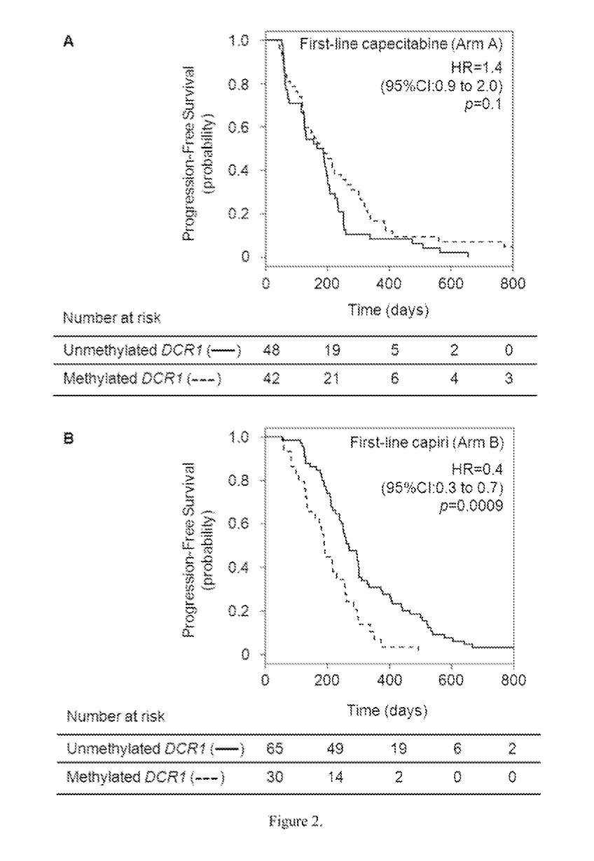

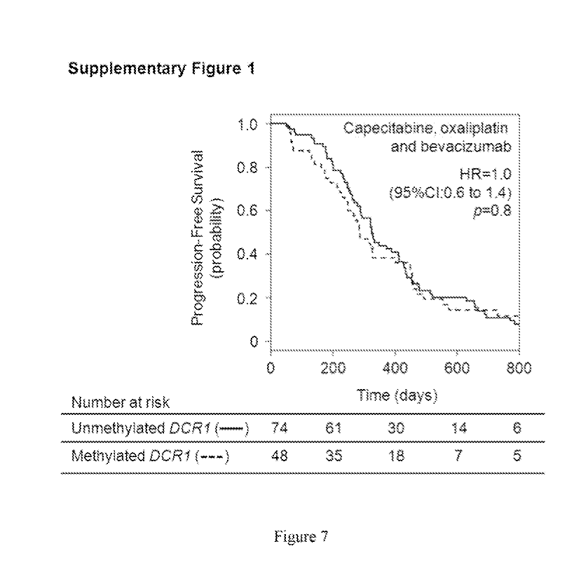

From these 22 genes, DCR1 (decoy receptor 1, also known as TNFRSF10C) showed the strongest correlation between methylation and outcome with respect to progression-free survival (PFS) (table 2). DCR1 was methylated in 72/185 (39%) tumors. Patients in arm B (first-line treatment with capiri) showed a significant shorter PFS when DCR1 was methylated compared to patients with unmethylated DCR1 (HR=0.4 (95% CI 0.3-0.7), p=0.0009; FIG. 2). This correlation was independent of clinical parameters like prior adjuvant treatment (p=0.7), predominant localization of metastases (p=0.6), serum LDH (p=0.4), WHO performance status (p=0.5), and age (p=0.2). In contrast, PFS for patients in arm A (treatment with capecitabine alone) was not significantly associated with methylation status (HR=1.4 (95% CI 0.9-2.0), p=0.1; see FIG. 7).

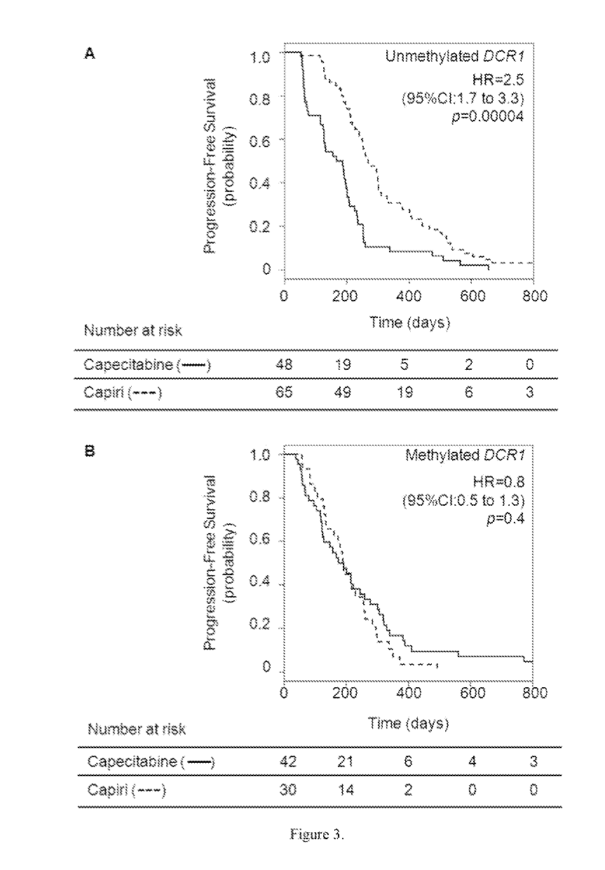

Like in the full CAIRO study population, for the 185 of patients from CAIRO in the discovery set, progression-free survival (PFS) was significantly longer for patients that received capiri (arm B) compared to patients that received capecitabine alone (arm A) (HR=1.5 (95% CI 1.1-2.0, p=0.01). However, when stratifying patients for DCR1 methylation status, patients with methylated DCR1 did not benefit from adding irinotecan to capecitabine (PFS arm B vs arm A: HR=0.8 (95% C/0.5-1.3, p=0.4). In contrast, patients with unmethylated DCR1 showed a significantly longer PFS when treated with capiri compared to capecitabine alone (PFS arm B vs arm A: HR=2.5 (95% CI 1.7-3.3, p=0.00004) with a median PFS benefit of 3 months (FIG. 3).

### Validation Set

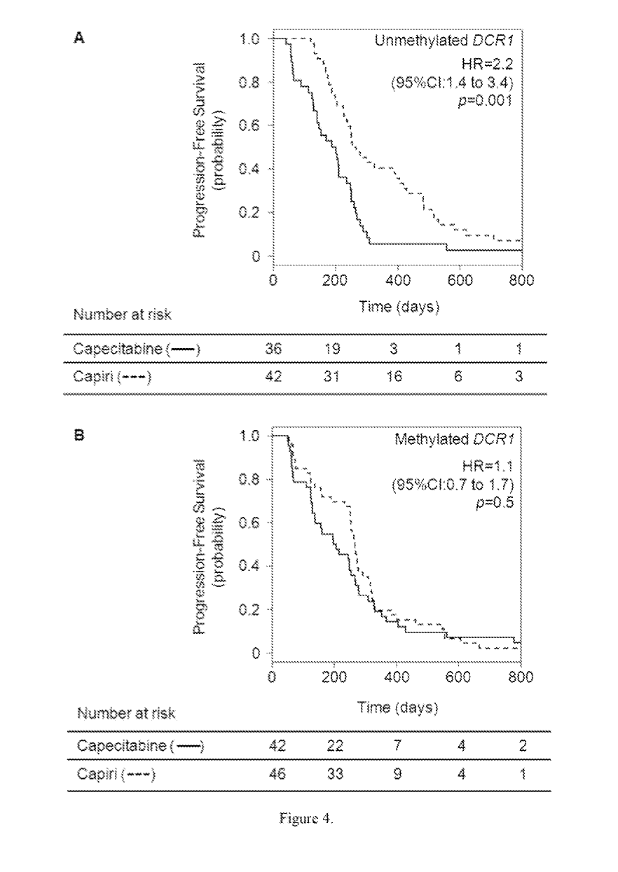

In order to validate methylated DCR1 as a marker for lack of response to irinotecan, a second set of patients from the CAIRO study was examined for tumor DCR1 methylation status and PFS. DCR1 was methylated in 88/166 (53%) tumors. Also in this series, overall PFS was significantly longer for patients treated with capiri (arm B) compared to patients treated with capecitabine alone (HR=1.7 (95% CI 1.1-2.0, p=0.004), but also here, after stratification for (DCR1 tumor methylation status, only a significant effect remained in patients with unmethylated DCR1 (HR=2.0 (95% CI 1.4-3.3, p=0.001) versus HR=1.1 (95% CI 0.7-1.7, p=0.6) for unmethylated and methylated tumor DCR1, respectively (see FIG. 4)). In the validations set the difference in median PFS was 2.2 months.

### Methylation of DCR1 is Associated to Decreased Gene Expression

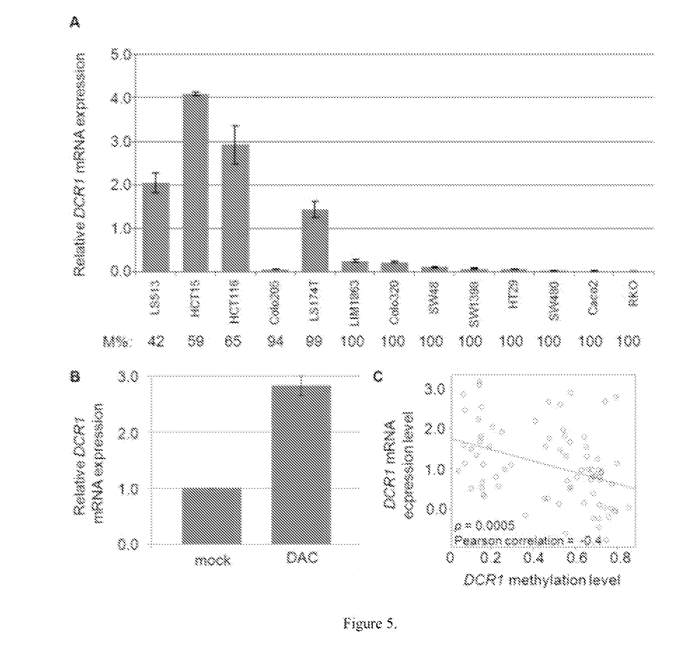

Hypermethylation of DCR1 resulting in down regulation of gene expression has been described in several cancer types (Shivapurkar N et al., Int J Cancer 2004; 109:786-92; van Noesel M M et al., Cancer Res 2002; 62:2157-61; Murphy T M et al., Prostate 2011; 71:1-17). To investigate the effect of methylation on expression in CRC, we investigated the association of DNA methylation measured by qMSP with mRNA expression measured by qRT-PCR for DCR1 in a panel of 13 CRC cell lines. Ten out of 13 CRC cell lines were fully methylated for DCR1 and showed low or absent gene expression. The other three CRC cell lines were hemi-methylated and showed clearly higher gene expression levels (FIG. 5A). Treatment of HCT116 (65% methylated for DCR1) with the demethylating agent 5-aza-2′-deoxycytidine (DAC) resulted in significant increased DCR1 expression (p=0.005: FIG. 5B). In addition, data from The Cancer Genome Atlas (TCGA) database (http://cancergenome.nih.gov), including 78 CRC tumors, confirmed a negative correlation between DCR1 methylation and mRNA expression in CRC (Pearson correlation of −0.4, p=0.0005; FIG. 5C).

### Discussion

Colorectal cancer biologically is a heterogeneous disease and much of this biological diversity is defined at the DNA level (mutations, copy number changes and promoter hypermethylation), giving rise to phenotypical differences and differences in clinical behavior, including risk of metastasis and response to drug therapy. The panel of anti-cancer drugs available for colorectal cancer has grown over the last two decades, providing now multiple options to the individual patient both for adjuvant treatment and systemic treatment of metastatic disease. While most of the drugs available for colorectal cancer are registered as one size fits all, given their different modes of action it is evident that differences in biology may affect response to these drugs. In the present study we used a candidate gene approach to test whether promoter hypermethylation status of a series of candidate genes, based on their function in relation to the mode of action of irinotecan, i.e. topoisomerase I inhibition, can predict response to first-line capiri treatment in patients with metastatic colorectal cancer. The present study was conducted using primary CRC tissues samples from a sub-set of patients from the Dutch CAIRO study. This study had two treatment arms, with first-line capecitabine in arm A and first-line capecitabine combined with irinotecan (capiri) in arm B (FIG. 1).

Patients with DCR1 methylated in their tumor did not benefit from the addition of irinotecan to capecitabine, in strong contrast to patients with unmethylated DC(RI in their tumor. The initial finding in the discovery set could be confirmed in a second series of patients from the same CAIRO study. The fact that in 65 patients analyzed from arm A for their response to single agent irinotecan therapy in second line, a similar trend was observed, although not statistically significant (data not shown), as well as the association between DCR1 methylation and mRNA expression from the TCGA data lends further support to this finding.

Because patients treated with capecitabine alone were used as a control group, DCR1 methylation could be considered to have a negative predictive value for response to irinotecan. Given the fact that the prevalence of DCR1 promoter hypermethylation overall is 46%, this finding is relevant for a large number of patients.

DCR1 is a decoy receptor for tumor necrosis factor (TNF) related apoptosis inducing ligand (TRAIL), which is part of the extrinsic apoptosis-signaling pathway. DCR1 is able to bind TRAIL, but fails to induce apoptosis since it lacks an intracellular death domain, and thus can act as a scavenger (Mahalingam D et al., Cancer Treat Rev 2009; 35:280-8). However, the role of TRAIL in regulating apoptosis is complex, as recently has been demonstrated. Next to tumor suppressor, i.e. pro-apoptotic, functions of TRAIL, it may also have oncogenic activity under certain circumstances, by activating NFkB, PI3K-Akt and other signal transduction pathways (Mellier G et al., Mol Aspects Med 201031:93-112; Verbrugge I et al., Cell 2010; 1192. e1-2; Johnstone R W et al., Nat Rev Cancer 2008; 8:782-98). Against that background, the frequently observed downregulation of DCRs in various cancers makes sense. Downregulation of DCR1 has been associated with DNA hypermethylation in different tumor types (Shivapurkar N et al., Int J Cancer 2004;109:786-92: van Noesel M M et al., Cancer Res 2002; 62:2157-61). In the present study, we identified DCR1 as a novel hypermethylated gene in CRC, with a frequency of 46%. The results on CRC cell lines in the present study and data on CRC tissue samples from the TCGA database suggest regulation of DCR1 expression by DNA methylation in CRC.

Knowing a priori that patients do not benefit from capiri over capecitabine alone may help reducing unnecessary toxicity for those patients, but then it is important to know whether alternative treatment modalities, e.g. oxaliplatin, would not be associated as well with DCR1 methylation status. We therefore tested for the association of response with DCR1 methylation status in primary tumor samples of 139 patients from the CAIROII phase III clinical trial treated with capecitabine, oxaliplatin and bevacizumab (capox-b) (Tol J et al., N Engl J Med 2009; 360:563-72). Indeed, PFS did not differ significantly between patients with methylated and unmethylated tumor DCR1 (FIG. 7), suggesting capox-based therapies indeed to be an alternative for patients with DCR1 methylated CRC. In the same line, patients with methylated DCR1 that fail first-line capox-based therapy will probably not benefit from second-line capiri-based therapy.

Interestingly, median PFS of patients with DCR1 unmethylated tumors was 8.9 months in the CAIRO study compared to 10.7 months in the CAIRO II study. Given the fact that CAIRO II patients also experienced a survival benefit from bevacuzimab, which can be estimated to be about two months (Tol J et al., N Engl J Med 2009; 360:563-72; Giantonio B J et al., J Clin Oncol 2007; 25:1539-44), given the data from the present study, capiri-based therapy in patients with DCR1 unmethylated tumors potentially is a very effective approach.

In conclusion, the present study revealed DCR1 methylation as a novel hypermethylated gene in CRC and as a predictive marker for lack of benefit from capiri over the single agent capecitabine in metastatic colorectal cancer in both the discovery and the validation set. These findings indicate a potential clinical relevance of DCR1 methylation status as a guide for selecting patients for treatment with irinotecan-based therapy.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method of assessing/determining/detecting expression or the methylation status of the DCR1 gene for predicting a clinical response to treatment of colon cancer, or for identifying and/or selecting a patient with colon cancer suitable for treatment, or for selecting a suitable treatment in a patient suffering from cancer wherein the treatment involves a thymidylate synthase inhibitor, a topoisomerase I inhibitor and/or a combination of the topoisomerase I inhibitor and the thymidylate synthase inhibitor.

2. A method of predicting a clinical response to treatment of colon cancer with a thymidylate synthase inhibitor, a topoisomerase I inhibitor and/or the combination of a topoisomerase I inhibitor and a thymidylate synthase inhibitor according to claim 1, comprising:
obtaining a biological sample from a patient,
assessing/determining/detecting in the sample expression or the methylation status of DCR1, and
predicting a benefit from treatment with the topoisomerase I or the combination of the topoisomerase I inhibitor and the thymidylate synthase inhibitor over the single agent thymidylate synthetase inhibitor if expression or a higher level of expression, or absence of methylation or a lower level of methylation of DCR1 is determined or detected,
or
predicting a lack of benefit from treatment with the topoisomerase I inhibitor or the combination of the topoisomerase I inhibitor and the thymidylate synthase inhibitor over the single agent thymidylate synthetase inhibitor if absence of expression or a lower level of expression, or methylation or a higher level of methylation of DCR1 is determined or detected.

3. (canceled)

4. A method for identifying and/or selecting a patient with colon cancer suitable for treatment according to claim 1, comprising:
obtaining a biological sample from the patient,
assessing/determining/detecting in the sample expression or the methylation status of DCR1 and/or regulatory regions thereof, and
identifying and/or selecting the patient for treatment with the topoisomerase I inhibitor or the combination of the topoisomerase I inhibitor and the thymidylate synthase inhibitor over the single agent thymidylate synthetase inhibitor if expression or a higher level of expression, or absence of methylation or a lower level of methylation of DCR1 is determined or detected,
or
identifying and/or selecting the patient as not suitable for treatment with the topoisomerase I inhibitor or the combination of the topoisomerase I inhibitor and the thymidylate synthase inhibitor over the single agent thymidylate synthetase inhibitor if absence of expression or a lower level of expression, or methylation or a higher level of methylation of DCR1 is determined or detected.

5. (canceled)

6. A method for selecting a suitable treatment in a patient suffering from colon cancer according to claim 1 comprising:
obtaining a biological sample from the patient,
assessing/determining/detecting in the sample expression or the methylation status of DCR1 and/or regulatory regions thereof, and
selecting the topoisomerase I inhibitor or the combination of the topoisomerase I inhibitor and the thymidylate synthase inhibitor over the single agent thymidylate synthetase inhibitor as the treatment if expression or a higher level of expression, or absence of methylation or a lower level of methylation of DCR1 is determined or detected,
or
selecting the single agent thymidylate synthetase inhibitor over the topoisomerase I inhibitor or the combination of the topoisomerase I inhibitor and the thymidylate synthase inhibitor as the treatment if absence of expression or a lower level of expression, or methylation or a higher level of methylation of DCR1 is determined or detected.

7.-10. (canceled)

11. A method comprising: (a) requesting a test providing results of an analysis to determine the methylation status of the gene DCR1 and its regulatory regions in a biological sample obtained from a patient; and (b) administering a thymidylate synthase inhibitor, a topoisomerase I inhibitor and/or a combination of the topoisomerase I inhibitor and the thymidylate synthase inhibitor based on the results of the test.

12. The method of claim 11, wherein the results of the test indicate whether DCR1, and its regulatory regions are nonmethylated, methylated, or hypermethylated.

13. The method of claim 11, wherein the results of the test indicate whether DCR1 and its regulatory regions are exhibiting a lower level of methylation or a higher level of methylation relative to a control.

14. The method of claim 11, comprising administering the topoisomerase I inhibitor and/or the combination of the topoisomerase I inhibitor and the thymidylate synthase inhibitor if the gene is nonmethylated or if the gene is exhibiting a lower level of methylation relative to a control,
or
comprising administering the thymidylate synthase inhibitor if the gene is methylated or if the gene is exhibiting a higher level of methylation relative to a control.

15. (canceled)

16. A method comprising: (a) requesting a test providing results of an analysis to determine the expression status of the DCR1 in a biological sample obtained from a patient; and (b) administering a thymidylate synthase inhibitor, a topoisomerase I inhibitor and/or a combination of the topoisomerase I inhibitor and the thymidylate synthase inhibitor based on the results of the test.

17. The method of claim 16, wherein the results of the test indicate whether DCR1 is expressed or is not expressed.

18. The method of claim 16, wherein the results of the test indicate whether DCR1 is expressed at a lower level or is expressed at a higher level relative to a control.

19. The method of claim 16, comprising administering a topoisomerase I inhibitor and/or a combination of a topoisomerase I inhibitor and a thymidylate synthase inhibitor if the gene is expressed or if the gene is expressed at a higher level relative to a control,
or
comprising administering a thymidylate synthase inhibitor if the gene is not expressed or if the gene is expressed at a lower level relative to a control.

20.-22. (canceled)

23. The method according to claim 1, wherein the thymidylate synthetase inhibitor is capecitabine.

24. The method according to claim 1, wherein the topoisomerase I inhibitor is irinotecan.

25. The method according to claim 1, wherein the combination of the topoisomerase I inhibitor and the thymidylate synthase inhibitor is capiri.

26. The method according to claim 1, wherein the gene is DCR1 and treatment is a combination of capecitabine and irinotecan.

27.-34. (canceled)

35. A primer or primer pair for determining the methylation status of DCR1 and/or regulatory regions thereof wherein the primer or primer pair comprises the nucleotide sequence or sequences set forth in Table 6.

36. A kit for assessing methylation in a test sample, comprising in a package:
a reagent that (a) modifies methylated cytosine residues but not non-methylated cytosine residues, or that (b) modifies non-methylated cytosine residues but not methylated cytosine residues; and
one or more oligonucleotide primers and/or pair of oligonucleotide primers that specifically hybridizes under amplification conditions to DCR1 and/or regulatory regions thereof, the one or more oligonucleotide primers and/or pair of oligonucleotide primers comprising the primer or primer Pair of claim 35.

37. (canceled)
